(19) United States
(12) Patent Application Publication
(10) Pub. No.: US 2023/0111350 A1
(43) Pub. Date: Apr. 13, 2023

(54) Opthalmic Compositions for Treating Coronaviruses

(71) Applicant: Minas Theodore Coroneo, Vaucluse, New South Wales (AU)

(72) Inventor: Minas Theodore Coroneo, Vaucluse, New South Wales (AU)

(21) Appl. No.: 17/913,712

(22) PCT Filed: Mar. 30, 2021
(86) PCT No.: PCT/IB2021/052627

§ 371 Date: Sep. 22, 2022

Related U.S. Application Data
(60) Provisional application No. 63/003,518, filed on Apr. 1, 2020.

(51) Int. Cl. A61K 31/4706; A61K 33/30; A61K 31/7052; A61K 9/00; A61K 31/737; A61K 31/4174; A61K 38/55; A61K 31/5415; A61K 31/451; A61K 31/496; A61K 31/513; A61K 31/135; A61K 31/497

(52) U.S. Cl. CPC A61K 31/4706; A61K 9/0014; A61K 31/135; A61K 31/451; A61K 31/496; A61K 31/497; A61K 31/513; A61K 31/737; A61K 31/4174; A61K 31/5415; A61K 31/7052; A61K 33/30; A61K 38/556

(57) ABSTRACT

The present invention relates to the field of viral infection therapeutic treatments and prophylactic treatments. In particular, provided are methods of treating or preventing viral infections, such as viral infections resulting from entering or infecting a subject via association with or binding to an ACE2 receptor in the subject, with ophthalmic composition, inhalation compositions, and/or topical compositions. In particular, provided are methods of treating or preventing viral infections, such as coronavirus infections, for example COVID-19 infections, in a subject exposed to, or prior to exposure to a coronavirus, for example a COVID-19, by administering an ophthalmic composition, an inhalation composition, and/or a topical composition.

### CROSS REFERENCE TO RELATED APPLICATION

This application claims the benefit of priority from U.S. Provisional Application No. 63/003,518, filed Apr. 1, 2020. The foregoing related application, in its entirety, is incorporated herein by reference.

### FIELD OF THE INVENTION

The present invention relates to the field of viral infection therapeutic treatments and prophylactic treatments. In particular, provided are methods of treating or preventing viral infections, such as viral infections resulting from entering or infecting a subject via association with or binding to an ACE2 receptor in the subject, with ophthalmic composition, inhalation compositions, and/or topical compositions. In particular, provided are methods of treating or preventing viral infections, such as coronavirus infections, for example COVID-19 infections, with ophthalmic composition, inhalation compositions, and/or topical compositions.

### BACKGROUND

The current coronavirus disease 2019 (also known as “COVID-19” and “2019-nCov”) epidemic, the third involving the zoonotic coronaviruses in as many decades (1) has bought to focus mechanisms of how this group of viruses spread (including human-to-human transmission) and how to prevent it (1). Superspreading (transmission of severe acute respiratory syndrome (SARS) to at least eight contacts (2)) may be critical (1, 2). A very recent study provides evidence for human-to-human transmission (3) and in question is how this could come about. Pathways include direct transmission, such as cough, sneeze, droplet inhalation transmission as well as contact transmission including contact with oral, nasal, and eye mucous membranes (4). The virus has been isolated in urine, stool and saliva (4, 5). However, the eye and its adnexae may play an unsuspected but significant role in disease transmission. The location of human eyes in the human body simultaneously provides information from our highest band width sense but also brings increased risk of exposure to the elements, including airborne viral particles.

### SUMMARY

Provided herein are a composition, comprising: i) an effective amount of hydroxychloroquine or chloroquine, or a pharmaceutically acceptable salt thereof; ii) an effective amount of a source of zinc ion; and iii) a pharmaceutically acceptable excipient, carrier or diluent; wherein the composition is an ophthalmic, an inhalation, or a topical composition. In some embodiments, the composition further comprises an effective amount of an ACE2 inhibitor. In some embodiments, the composition further comprises an effective amount of a vasoconstrictor. In some embodiments, the composition further comprises an effective amount of iota-carrageenan or a pharmaceutically acceptable salt thereof.

Provided herein are also a composition, comprising: i) an effective amount of hydroxychloroquine or chloroquine, or a pharmaceutically acceptable salt thereof; ii) an effective amount of azithromycin; and iii) a pharmaceutically acceptable excipient, carrier or diluent; wherein the composition is an ophthalmic, an inhalation, or a topical composition. In some embodiments, the composition further comprises an effective amount of an ACE2 inhibitor. In some embodiments, the composition further comprises an effective amount of a vasoconstrictor. In some embodiments, the composition further comprises an effective amount of iota-carrageenan or a pharmaceutically acceptable salt thereof.

Provided herein are also a composition, comprising: i) an effective amount of hydroxychloroquine or chloroquine, or a pharmaceutically acceptable salt thereof; ii) an effective amount of a source of zinc ion; iii) an effective amount of azithromycin; and iv) a pharmaceutically acceptable excipient, carrier or diluent; wherein the composition is an ophthalmic, an inhalation, or a topical composition. In some embodiments, the composition further comprises an effective amount of an ACE2 inhibitor. In some embodiments, the composition further comprises an effective amount of a vasoconstrictor. In some embodiments, the composition further comprises an effective amount of iota-carrageenan or a pharmaceutically acceptable salt thereof.

Provided herein are also a method of treating a subject exposed to a virus, comprising administering to a subject (e.g., a human) exposed to a virus an effective amount of a composition, comprising: i) an effective amount of hydroxychloroquine or chloroquine, or a pharmaceutically acceptable salt thereof; ii) an effective amount of a source of zinc ion; and iii) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of treating a subject exposed to a virus, comprising administering to a subject exposed to a virus an effective amount of a composition, comprising: i) an effective amount of hydroxychloroquine or chloroquine, or a pharmaceutically acceptable salt thereof; ii) an effective amount of azithromycin; and iii) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of treating a subject exposed to a virus, comprising administering to a subject exposed to a virus an effective amount of a composition, comprising: i) an effective amount of hydroxychloroquine or chloroquine, or a pharmaceutically acceptable salt thereof; ii) an effective amount of a source of zinc ion; iii) an effective amount of azithromycin; and iv) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of treating a subject exposed to a virus, comprising administering to a subject exposed to a virus an effective amount of a composition, comprising: i) an effective amount of iota-carrageenan or a pharmaceutically acceptable salt thereof; and ii) a pharmaceutically acceptable excipient, carrier or diluent; wherein the composition is an ophthalmic, an inhalation, or a topical composition. In some embodiments, the administered composition comprises: i) an effective amount of iotacarrageenan or a pharmaceutically acceptable salt thereof; ii) an effective amount of hydroxychloroquine or chloroquine, or a pharmaceutically acceptable salt thereof; and iii) a pharmaceutically acceptable excipient, carrier or diluent; wherein the composition is an ophthalmic, an inhalation, or a topical composition. In some embodiments, the administered composition comprises: i) an effective amount of iota-carrageenan or a pharmaceutically acceptable salt thereof; ii) an effective amount of hydroxychloroquine or chloroquine, or a pharmaceutically acceptable salt thereof; iii) an effective amount of azithromycin; and iv) a pharmaceutically acceptable excipient, carrier or diluent; wherein the composition is an ophthalmic, an inhalation, or a topical composition. In some embodiments, the administered composition comprises: i) an effective amount of iota-carrageenan or a pharmaceutically acceptable salt thereof; ii) an effective amount of hydroxychloroquine or chloroquine, or a pharmaceutically acceptable salt thereof; iii) an effective amount of azithromycin; iv) an effective amount of a source of zinc ion; and v) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of preventing or treating a virus infection in a subject, comprising administering to a subject exposed to a virus an effective amount of a composition, comprising: i) an effective amount of hydroxychloroquine or chloroquine, or a pharmaceutically acceptable salt thereof; ii) an effective amount of a source of zinc ion; and iii) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of preventing or treating a virus infection in a subject, comprising administering to a subject exposed to a virus an effective amount of a composition, comprising: i) an effective amount of hydroxychloroquine or chloroquine, or a pharmaceutically acceptable salt thereof; ii) an effective amount of azithromycin; and iii) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of preventing or treating a virus infection in a subject, comprising administering to a subject exposed to a virus an effective amount of a composition, comprising: i) an effective amount of hydroxychloroquine or chloroquine, or a pharmaceutically acceptable salt thereof; ii) an effective amount of a source of zinc ion; iii) an effective amount of azithromycin; and iv) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of preventing or treating a virus infection in a subject, comprising administering to a subject exposed to a virus an effective amount of a composition, comprising: i) an effective amount of iota-carrageenan or a pharmaceutically acceptable salt thereof; and ii) a pharmaceutically acceptable excipient, carrier or diluent; wherein the composition is an ophthalmic, an inhalation, or a topical composition. In some embodiments, the administered composition comprises: i) an effective amount of iotacarrageenan or a pharmaceutically acceptable salt thereof; ii) an effective amount of hydroxychloroquine or chloroquine, or a pharmaceutically acceptable salt thereof; and iii) a pharmaceutically acceptable excipient, carrier or diluent; wherein the composition is an ophthalmic, an inhalation, or a topical composition. In some embodiments, the administered composition comprises: i) an effective amount of iota-carrageenan or a pharmaceutically acceptable salt thereof; ii) an effective amount of hydroxychloroquine or chloroquine, or a pharmaceutically acceptable salt thereof; iii) an effective amount of azithromycin; and iv) a pharmaceutically acceptable excipient, carrier or diluent; wherein the composition is an ophthalmic, an inhalation, or a topical composition. In some embodiments, the administered composition comprises: i) an effective amount of iota-carrageenan or a pharmaceutically acceptable salt thereof; ii) an effective amount of hydroxychloroquine or chloroquine, or a pharmaceutically acceptable salt thereof; iii) an effective amount of azithromycin; iv) an effective amount of a source of zinc ion; and v) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of prophylactically treating, comprising administering to a subject prior to exposure to a virus an effective amount of a composition, comprising: i) an effective amount of hydroxychloroquine or chloroquine, or a pharmaceutically acceptable salt thereof; ii) an effective amount of a source of zinc ion; and iii) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of prophylactically treating, comprising administering to a subject prior to exposure to a virus an effective amount of a composition, comprising: i) an effective amount of hydroxychloroquine or chloroquine, or a pharmaceutically acceptable salt thereof; ii) an effective amount of azithromycin; and iii) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of prophylactically treating, comprising administering to a subject prior to exposure to a virus an effective amount of a composition, comprising: i) an effective amount of hydroxychloroquine or chloroquine, or a pharmaceutically acceptable salt thereof; ii) an effective amount of a source of zinc ion; iii) an effective amount of azithromycin; and iv) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the administered composition further comprises an effective amount of iota-carrageenan or a pharmaceutically acceptable salt thereof. In some embodiments, the virus is a virus that enters or infects a subject via association with or binding to an ACE2 receptor in the subject. In some embodiments, the virus is a coronavirus. In some embodiments, the coronavirus is COVID-19.

Provided herein are also a method of prophylactically treating, comprising administering to a subject prior to exposure to a virus an effective amount of a composition, comprising: i) an effective amount of iota-carrageenan or a pharmaceutically acceptable salt thereof; and ii) a pharmaceutically acceptable excipient, carrier or diluent; wherein the composition is an ophthalmic, an inhalation, or a topical composition. In some embodiments, the administered composition comprises: i) an effective amount of iota-carrageenan or a pharmaceutically acceptable salt thereof; ii) an effective amount of hydroxychloroquine or chloroquine, or a pharmaceutically acceptable salt thereof; and iii) a pharmaceutically acceptable excipient, carrier or diluent; wherein the composition is an ophthalmic, an inhalation, or a topical composition. In some embodiments, the administered composition comprises: i) an effective amount of iota-carrageenan or a pharmaceutically acceptable salt thereof; ii) an effective amount of hydroxychloroquine or chloroquine, or a pharmaceutically acceptable salt thereof; iii) an effective amount of azithromycin; and iv) a pharmaceutically acceptable excipient, carrier or diluent; wherein the composition is an ophthalmic, an inhalation, or a topical composition. In some embodiments, the administered composition comprises: i) an effective amount of iota-carrageenan or a pharmaceutically acceptable salt thereof; ii) an effective amount of hydroxychloroquine or chloroquine, or a pharmaceutically acceptable salt thereof; iii) an effective amount of azithromycin; iv) an effective amount of a source of zinc ion; and v) a pharmaceutically acceptable excipient, carrier or diluent; wherein the composition is an ophthalmic, an inhalation, or a topical composition. In some embodiments, the administered composition further comprises an effective amount of an ACE2 inhibitor. In some embodiments, the administered composition further comprises an effective amount of a vasoconstrictor. In some embodiments, the virus is a virus that enters or infects a subject via association with or binding to an ACE2 receptor in the subject. In some embodiments, the virus is a coronavirus. In some embodiments, the coronavirus is COVID-19.

### BRIEF DESCRIPTION OF THE DRAWINGS

FIG. 1. Graphic illustrating the relatively large surface area of the eye(s) was compared to that of the mouth and nares.

FIG. 2. Illustration of the lacrimatory-nasal mechanism for the mechanical disposition of organisms entering the upper respiratory tract.

FIG. 3. Illustration showing the superficial tear film lipid layer and the structures involved in the production of tear film.

### DETAILED DESCRIPTION

Provided herein are an ophthalmic, an inhalation, or a topical composition, and methods of using the same, for treatment and/or prevention of a virus infection in a subject exposed to, or prior to exposure to, a virus. In some embodiments, the virus is a virus that enters or infects a subject via association with or binding to an ACE2 receptor in the subject. In some embodiments, the virus is a coronavirus. In some embodiments, the coronavirus is COVID-19. In some embodiments, the virus is influenza. In some embodiments, the virus is a filovirus. In some embodiments, the virus is ebola.

The subject can be a mammal, for example, a human. The subject can be male or female, and can be an adult, child or infant. The subject can be a patient who has a virus infection (e.g., has been diagnosed with a virus infection), or the subject can be a patient who has been exposed to a virus, but does not show one or more symptoms of having a virus infection. In some embodiments, the virus is a virus that enters or infects a subject via association with or binding to an ACE2 receptor in the subject. In some embodiments, the virus is a coronavirus. In some embodiments, the coronavirus is COVID-19.

### Definitions

As used herein, the articles “a,” “an,” and “the” refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof.

As used herein, the term “sample” refers to a material or mixture of materials containing one or more components of interest. A sample from a subject refers to a sample obtained from the subject, including samples of biological tissue or fluid origin, obtained, reached, or collected in vivo or in situ. A sample can be obtained from a region of a subject containing precancerous or cancer cells or tissues. Such samples can be, but are not limited to, organs, tissues, fractions and cells isolated from a mammal. Exemplary samples include saliva, lymph node, whole blood, partially purified blood, serum, peripheral blood, a cell lysate, a cell culture, a cell line, a tissue, oral tissue, gastrointestinal tissue, an organ, an organelle, a biological fluid, a blood sample, a urine sample, a skin sample, and the like.

As used herein, the term “treat,” “treating,” and “treatment,” when used in reference to a virus infected patient, refer to an action that reduces the severity of the virus infection, or retards or slows the progression of the virus infection, including (a) inhibiting the viral replication of the virus in the subject, and (b) causing shedding of the viral load, or delaying or minimizing one or more symptoms associated with the virus infection. In some embodiments, the virus is a virus that enters or infects a subject via association with or binding to an ACE2 receptor in the subject. In some embodiments, the virus is a coronavirus. In some embodiments, the coronavirus is COVID-19. In some embodiments, the virus is influenza. In some embodiments, the virus is a filovirus. In some embodiments, the virus is ebola.

As used herein, the term “administer,” “administering,” or “administration” refers to the act of delivering, or causing to be delivered, a compound or a pharmaceutical composition to the body of a subject by a method described herein or otherwise known in the art.

As used herein, the term “therapeutically effective amount” of a compound or a composition when used in connection with a disease or disorder, such as an infection, for example a viral infection, refers to an amount sufficient to provide a therapeutic benefit in the treatment or management of the disease or disorder or to delay or minimize one or more symptoms associated with the disease or disorder. A therapeutically effective amount of a compound or a composition means an amount of the compound or the composition that when used alone or in combination with other therapies, would provide a therapeutic benefit in the treatment or management of the disease or disorder. The term encompasses an amount that improves overall therapy, reduces or avoids symptoms, or enhances the therapeutic efficacy of another therapeutic agent. The term also refers to the amount of a compound that sufficiently elicits the biological or medical response of a biological molecule (e.g., a protein, enzyme, RNA, or DNA), cell, tissue, system, animal, or human, which is being sought by a researcher, veterinarian, medical doctor, or clinician.

### Ocular System and Viral Infection

A cornavirus, such as the COVID-19 virus, can be a highly contagious, oculotropic, respiratory virus associated with a relatively high mortality rate. The eye and its adnexae represent a large surface area with direct exposure to droplets as well as to indirect, hand contact. The cornea and conjunctiva contain angiotensin converting enzyme 2 (ACE2) receptors to which the virus binds and the lipophilic periocular skin and superficial tear film are also likely to bind the virus with resultant carriage into the nasopharynx with access to both the lungs and gastrointestinal tract. This pathway however can be accessed by eye drops or aerosols containing drugs which appear to work via systemic administration. Of these, in some embodiments, a composition as disclosed here, such as an ophthalmic composition, an inhalation composition, or a topical composition, and methods of using the same, comprising hydroxychloroquine or a pharmaceutically acceptable salt thereof, alone or in combination with azithromycin and/or a source of zinc ion, all of which have previously been used safely and directly on the eye, may offer a safe, cost-effective and resource-sparing intervention of a viral infection. In some embodiments, a composition as disclosed here, such as an ophthalmic composition, an inhalation composition, or a topical composition, and methods of using the same, comprising chloroquine or a pharmaceutically acceptable salt thereof, alone or in combination with azithromycin and/or a source of zinc ion, all of which have previously been used safely and directly on the eye, may offer a safe, cost-effective and resource-sparing intervention of a viral infection. In some embodiments, a composition as disclosed here, such as an ophthalmic composition, an inhalation composition, or a topical composition, and methods of using the same, comprising iota carrageenan or a pharmaceutically acceptable salt thereof, alone or in combination with hydroxychloroquine or chloroquine, or a pharmaceutically acceptable salt thereof, respectively, azithromycin, and/or a source of zinc ion, all of which have previously been used safely and directly on the eye, may offer a safe, cost-effective and resource-sparing intervention of a viral infection. In some embodiments, the ophthalmic composition, the inhalation composition, or the topical composition, as disclosed herein, is suitable for oral administration. In some embodiments, the compositions may optionally further comprise an ACE2 inhibitor (blocker). In some embodiments, the composition further comprises an effective amount of a vasoconstrictor. In some embodiments, the administered composition further comprises an effective amount of iotacarrageenan or a pharmaceutically acceptable salt thereof. Without being bound to theory, one or more of these agents may work, at least in part, by blocking ACE2 receptors, which may offer a safe, cost-effective and resource-sparing intervention of a viral infection. In some embodiments, the viral infection is a viral infection resulting from entering or infecting a subject via association with or binding to an ACE2 receptor in the subject. In some embodiments, the viral infection is a coronavirus infection. In some embodiments, the viral infection is a COVID-19 infection.

It is not well recognized that COVID-19 is one of the respiratory viruses that exhibit ocular tropism (6) and spread of disease via lacrimal tears and the ocular surface has been suspected (7). Despite early inconclusive evidence of coronavirus replication in ocular samples, several studies underscore the eye as a potential portal of entry for this virus (5-9). The virus has been associated with conjunctivitis (10-13). Human coronavirus NL 63 (HCoV-NL63) was initially identified in a baby with conjunctivitis and bronchiolitis (10) and subsequently, conjunctivitis was diagnosed in 17% of HCoV-NL63 infected children (11). As discussed further below however, conjunctivitis may not necessarily be a hallmark of viral entry via the ocular surface port.

### Role of the Ocular Surface

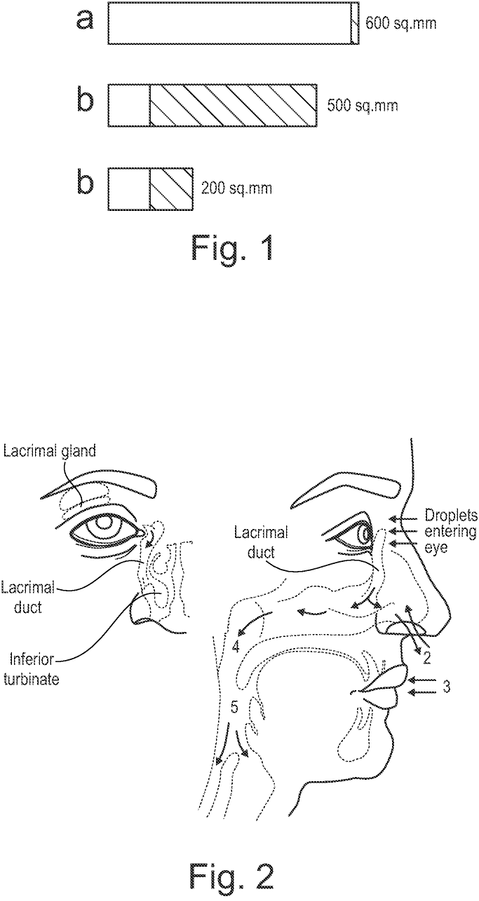

This potential mechanism of spread was recognized as early as 1919 (14), when it was pointed out that “the eye had received little or no attention as a factor in the transmission of acute respiratory infections. The preventable droplet spray means of contagious disease transmission, termed “promiscuous spraying” was known and coughing could project material at least 10 feet away. The relatively large surface area of the eye(s) was compared to that of the mouth and nares, and is shown in FIG. 1, illustrating the relative importance of eye exposure to direct droplet spray (wherein (a) represents average total eye surface exposed; shaded area represents proportion of time not exposed, due to winking; (b) represents average total mouth area exposed in talking; shaded area represents proportion of time not exposed, due to closure; and (c) represents average total area of cross-section of nares exposed; shaded area represents proportion of time not exposed, owing to protected position and expiration) (14).

Since this 1919 study (14), a number of other studies have investigated ocular surface area and reported as a total (for two eyes) as 226-426 (15) and 300-640 mm² (16), indicating that the figure from Maxcy’s early study (10) was a good estimate (summary of External Ocular Surface Areas is provided in Table 1) of the “visible” ocular surface. The later study (16) also helps to explain reasons for variability in measurements since palpebral aperture is dependant of position of eye gaze.

TABLE 1

External Ocular Surface Areas

| | Area (mm²) | Methodology | Reference |
|---|---|---|---|
| Palpebral Aperture | 600 | Unreported | 14 |
| Palpebral Aperture | 226-426 | Digitized video images | 15 |
| Palpebral Aperture | 300-640 | Digitized video images | 16 |
| Total Ocular Surface Area | 3200 | Designed instrument | 17 |
| Total Ocular Surface Area | 3738 | Molds of cadaver eyes | 18 |
| Total Orbital Aperture Area | 2509 (estimated) | CT Scan | 19 |
| Total Orbital Aperture Area | 1793-1987 | Radiographs | 20 |

However, the total ocular surface area have been estimated at ~1600-1869 mm²/eye (17,18), including the cornea, so for both eyes this represents a maximal absorptive area of ~3738 mm², accounting for the conjunctival lining of the eyelids, the tarsal conjunctiva and including the palpebral fornices. Even this consideration might underestimate the potential ocular/periocular landing zone for a viral particle. It is not an uncommon observation that makeup, applied around the eye, can “migrate” onto the ocular surface (21), noxious agent similar to the phenomenon by which noxious agents in minute quantities are easily transferred from fingertip to periocular skin and then the eye. It was hypothesized that this is due to subtle actions of muscles of Riolan (22). In fact, this mechanism of transport of agents from the periocular eye lid skin to the eye (obviating the need for eyedrops) has been termed supracutaneous and has been developed as an efficient delivery system for management of dry eye syndrome (23).

This supracutaneous mechanism, however might provide a substantial periocular area in which viral particles could land and be “funnelled” onto the ocular surface and beyond. Table 1 provides available information on the size of the orbital opening (19 and reviewed in 20), which likely underestimates the area of eyelid skin. We have estimated eyelid skin to be ~4000 mm² and that of the brow to be ~3000 mm², so that in total with the ocular surface area a landing site of ~10,000 mm² would be available, which is 2 orders of magnitude greater than for the nares and mouth. This does not take into account the surface area that could be attributed to the hair of the eyebrows or the eyelashes, for which estimates have not been made.

However, eyelash aerodynamics may play a role (24). Eyelashes have been shown to divert airflows, acting as a passive ocular dust controlling system. They reduce evaporation and particle deposition up to 50%. It has been postulated that the evolution of eyelashes may have played a role in reducing the endogenous blink rate. It appears that in modelling experiments, there is an ideal eyelash length in relation to retarding evaporation and that increasing length beyond this can increase evaporation (25). Optimal eyelash length is around 15-30% of eye width and can offer approximately 10-30% evaporation reduction as compared to the eyelash free state. Human eyelashes were judged to be longer than the “optimal” modelling length and enhanced particle capture was offered as an explanation. Furthermore, in a comparative study (26). Asian eyelashes had lower lift-up and curl-up angles, fewer numbers and a thicker transverse diameter as compared to Caucasian eyelashes. They did differ significantly in length or growth rate. Whether these differences play any role in influencing the rate at which particles land on the ocular surface is unknown. However, it is possible that a deficiency in this mechanism could increase the risk of infection.

In the 1919 study (14), Bacillus prodigiosus (Serratia marcescens) was instilled into the lacrimal sac of 5 volunteers and subsequently was recoverable from the nose, throat and stool after 5 mins, 15 mins and 24 hours respectively. It was concluded that via this ocular lacrimatory-nasal mechanism (shown in FIG. 2, excerpted from (14)), that the upper respiratory tract of a person wearing a properly constructed mask may be infected by exposing the eye briefly to a direct droplet spray.

### Viral Aerosolization

Viruses can be released into the air directly from animals by their breathing, coughing, and sneezing in droplets or by secondary aerosolization and that aerosolization has a critical impact on the aerodynamic size and particle behaviour (27). While particle size determines whether or not a particle can be inhaled and retained in the respiratory tract, this is not the case for alighting on the ocular surface. While transmission of coronaviruses occurs via an airborne route (28), because infected, symptomatic patients tend to develop severe lower as opposed to upper respiratory tract infections, it has been supposed that airborne agent virus has to be small enough to penetrate directly into the lower respiratory tract to preferentially replicate there before causing disease. That transmission could occur via the ocular-nasolacrimal pathway was not considered.

### The Preocular Tear Film

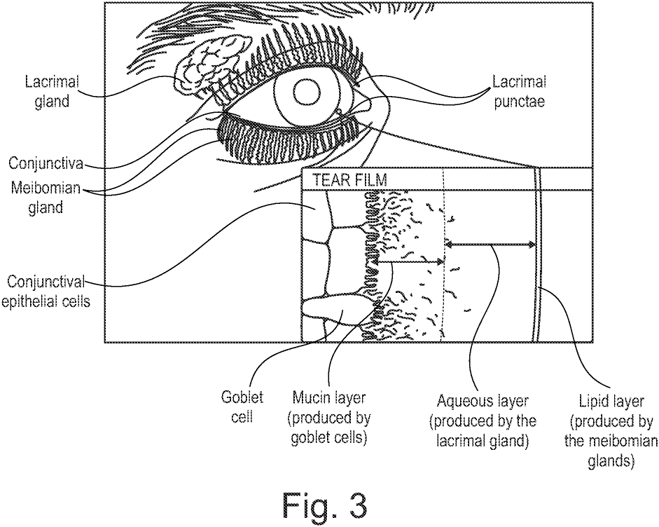

The layered tear film, the first refractive surface of the eye, has mechanical, optical, anti-microbial and nutritional properties and its dysfunction in dry eye syndrome has significant consequences for ocular and human health (29). The most superficial tear film lipid layer (as shown in FIG. 3, illustrating the structures involved in the production of tear film (excerpted from (29)) retards tear film evaporation and helps to prevent spillage over the eyelid margins.

However this lipophilicity and that of the periocular skin (30) may play a role in how coronaviruses and perhaps other enveloped viruses access the ocular surface. In the skin, extracellular surface lipids provide a barrier function (31) and while a similar function might keep viruses from accessing ocular surface receptors, tear and supracutaneous flow would result in a “second chance” for the virus to bind to receptors downstream in the lacrimal drainage pathway and beyond. The tear film, comprised of a nonpolar lipids is generally hydrophobic, though some lipids can bind with proteins (32). The coronaviruses with a crown of spike proteins around a lipid bilayer envelope (33) may be well suited to adhering to the ocular surface. Coronavirus cell entry and adhesion require lipid rafts and the presence of cell membrane cholesterol (34,35) and in a sense, the cholesterol containing tear lipid layer (36) might act as “lipid raft,” facilitating initial viral adhesion.

The ocular surface carries electrostatic charges - negatively charged mucins present in the tear film to which cationic oil-in-water nanoemulsions can bind (37). There appears to be little information on the surface charge of coronaviruses, either naked or in a bioaerosal. However, when ionisers are used to neutralize influenza viruses, negative ions are generated, rendering airborne particles/ aerosol droplets negatively charged thereby making them electrostatically attractive to a positively charged collector plate (38). This suggest that the viral particle may be positively charged. If this is the case, such viral particles might be attractive to the ocular surface. In fact, for the severe acute respiratory syndrome coronavirus (SARS-CoV), the surface charge distribution is pronouncedly asymmetrical (39). The flat surface of the helical sheet is large negatively charged region, adjacent to a large hydrophobic patch and on the opposite side there a positively charged groove that extends along the helix α1, a situation compatible with adherence to the tear film surface.

### Role of Ocular Surface Cellular Receptors

Identification of ocular surface cellular receptors utilized by respiratory viruses has provided information as to the permissiveness of ocular tissue to infection with these agents (6, 40). One possible path for 2019-nCov pathogenicity may be cellular entry via the cell surface angiotensin converting enzyme 2 (ACE2) receptor (41, 42, 125-127). It is the only mammalian group I coronavirus known to use angiotensin-converting enzyme 2 (ACE2) as its receptor (43). Recently, using spike protein modelling, the likely receptor for 2019-nCov has been identified as the Angiotensin-converting enzyme 2 (ACE2) (41). ACE2 was previously identified as the receptor for SARS-Cov and NL63 (43-45). Although the binding strength between 2019-nCov and ACE2 was modelled to be weaker than that between SARS-Cov and ACE2, it is much higher than the threshold required for virus infection. Virus infectivity studies have shown that ACE2 is essential for 2019- nCov to enter HeLa cells (46). These data have been interpreted to indicate that ACE2 may be the receptor for 2019-nCov (42).

While ACE2 mRNA is known to be present in virtually all organs, surface expression of ACE2 protein was described on lung alveolar epithelial cells and enterocytes of the small intestine (47). ACE2 was also present in arterial and venous endothelial cells and arterial smooth muscle cells in all organs studied. It has been postulated that ACE2 in the epithelia of the lung and small intestine might provide possible routes of entry for the coronavirus, but the eye and its adnexae were not investigated in this study. It was subsequently shown that ACE2 protein is more abundantly expressed on the apical than the basolateral surface of polarized airway epithelia (48). Furthermore, ACE2 expression positively correlated with the state of epithelial differentiation and susceptibility to infection, whereas well-differentiated cells expressing more ACE2 were readily infected with SARS-CoV, undifferentiated cells expressing little ACE2 were poorly infected (48). An immunohistochemical study revealed both extra- and intraocular localisation of ACE in human eyes (49). Of particular interest was the localisation of ACE to the epithelial cells of both the cornea and conjunctiva.

The resilience of the tear film may well protect the underlying corneal and conjunctival epithelium from coronaviruses. Adherence to the tear film by the virus may prevent access to the apical surface of the epithelial cells, but may also result in the virus being swept through the lacrimal puncta into the vast surface area of the lacrimal drainage system and beyond. This “wash-through” effect might account for the relatively low incidence of keratitis/conjunctivitis (10,11).

### The Nasolacrimal System

The lacrimal drainage system could be viewed as an extension of the ocular surface and its role has more recently been extensively reviewed (6). That the nasolacrimal system provides an anatomical bridge between ocular and respiratory tissues, serving as a conduit for virus-containing fluid exchange between these sites is reiterated. Furthermore, beyond the anatomical linkage of ocular and respiratory tract tissues, it is pointed out that the structure and distribution of cellular receptors in these systems is likely contribute to the tissue tropism of respiratory viruses (6). Furthermore, there is respiratory - ocular mucosal immune interdependence with linkage via the nasolacrimal lymphoid tissue (6, 50). Despite tear film antimicrobial peptides, several viral agents have been detected in the tear fluid of symptomatic, chronic, and asymptomatic individuals, confirming the potential for ocular involvement following viral respiratory and other infections (6, 51-56).

### Viral Transmission and Protection Guidelines

The present epidemic of 2019-nCov has raised particular interest in personperson viral transmission and mechanisms by which this might occur. Despite early suggestions of ocular involvement (5, 8-12) in this process, it appears that this was not taken into consideration in framing guidelines for protection against infection (57). While the use of masks is recommended, advice in relation the eyes is “avoid touching your eyes, nose, and mouth with unwashed hands.” However, in a health care setting dealing with infected patients or suspects, Interim Recommendations include eye protection (58). That this is not always the case is evidenced by recent events in Wuhan, China. As reported recently (59), while an N95 mask was worn by one of the earliest known individuals infected by 2019-nCoV in Wuhan (60), eye protection was not. Apparently, several days before the onset of pneumonia, the earliest symptoms shown by this earliest known infected individual were conjunctivitis of the left eye (60), then catarrhal symptoms and fever which developed after 2-3 hours, perhaps a little slower than earlier reported for the passage of bacteria in the lacrimal drainage system (14). Furthermore, it has also been reported that an ophthalmologist died following infection with 2019-nCoV (61). It was thought this ophthalmologist was infected during examination in the second week of January 2020, of a female patient with glaucoma. The proximity between an ophthalmologist and a patient during examinations may also increase the chance of contracting a higher initial viral nasopharyngeal load, including in the eyes, from an infected individual (either the ophthalmologist or the patient), which may lead to an increased severity of an eventual viral infection (129).

Based on present evidence, the Interim recommendations (58) are a reasonable approach, but are not the only method for providing prophylactic treatment, or to prevent the spread of and/or infections by the virus. For example, based on circumstantial and historical evidence, the ocular surface may well be an excellent portal for coronaviruses to enter the human body, since the ocular surface offers a large and receptor laden and lipophilic landing area for viral particles and the tear film and lacrimal drainage system offer a second opportunity for viral attachment and access to both respiratory and gastrointestinal systems. More recently, American Academy of Ophthalmology recommendations include protection for the mouth, nose and eyes when caring for patients potentially infected with this virus (62), reiterated in a recent commentary (63).

Mortality rates of 9.5% and 34.5% for earlier coronavirus outbreaks and 2.3% for the present (64), have been reported, however, a mortality rate of ~12% has been reported in the epidemic epicentre (65) but may be a s high as 20% (66). As of February 2020, the death toll from the COVID-19 epidemic caused by coronavirus SARS-CoV-2, has surpassed the combined death toll of the SARS (Severe Acute Respiratory Syndrome) epidemic of 2002-2003 and the MERS (Middle East Respiratory Syndrome) epidemic of 2013 combined (67). Until such time as this method of transmission is confirmed, re-evaluation of the ocular portal for transmission of this group of viruses is warranted, and eye protection may become an essential strategy in combating coronavirus epidemics. Accordingly, efficient and cost-effective methods of providing eye protection need to be developed.

### Compositions and the Uses Thereof in Prevention and/or Therapeutic Treatment of Viral Infection

The ocular surface, representing a large surface area, exposed to and likely receptive to coronavirus, may be an ideal point of intervention to prevent and/or treat a coronavirus infection. To date, a broad range of therapeutic options and targets for drug discovery have been pursued (68-73) in attempts to deal with coronavirus infection, but such options have been focused on systemic treatments, particularly orally administered treatments.

The ocular surface, bearing the appropriate ACE2 receptor, may be an ideal point of intervention, although to date, efforts in targeting the ACE2 receptor seem limited (74). For example, blocking the ACE2 receptor may deprive coronaviruses from their main point of tissue binding. For example, P4 and P5 peptides and NAAE, a small molecule targeting ACE2, have been developed but there have been concerns about a narrow spectrum of activity and effects on blood pressure regulation (67).

In some embodiments, the composition, such as an ophthalmic, an inhalation, or a topical composition, as disclosed herein, and the methods of using the same, may target the ACE2 receptor, and may include, but are not limited to, an ACE2 inhibitor and/or hydroxychloroquine (or chloroquine) or a pharmaceutically acceptable salt thereof. In some embodiments, the composition, such as an ophthalmic, an inhalation, or a topical composition, as disclosed herein, and the methods of using the same, may target the ACE2 receptor, and may include, but are not limited to, an ACE2 inhibitor and/or iota carrageenan or a pharmaceutically acceptable salt thereof. In some embodiments, the composition is an ophthalmic composition. In some embodiments, the composition is an inhalation composition. In some embodiments, the composition is a topical composition. In some embodiments, the ophthalmic composition, the inhalation composition, or the topical composition, is suitable for oral administration. ACE inhibitors are widely used in the treatment of systemic hypertension, congestive cardiac failure, diabetic nephropathy and are generally well tolerated (76). This class of drugs has not previously been considered as having an anti-viral role. Crystallography studies demonstrate that while the ACE inhibitor Lisinopril binds in a region near the centre of the receptor (77,78), the virus binding sites are on the outer surface of the receptor near the N terminal (43), so direct blocking by Lisinopril of the virus attachment site seems unlikely. However, the potent ACE2 inhibitor MLN-4760 induces a large receptor conformational change, a hinge bending motion, important for both inhibitor binding and catalysis, which may be unfavourable for viral binding to the receptor and/or syncytial formation (78). In some embodiments, ACE2 inhibitors, such as MLN-4760 or DX600, may be useful in preventing viral binding to the ACE2 receptor (79, 128).

FDA-approved drugs, such as chloroquine, chlorpromazine, loperamide and lopinavir, may have anti-coronavirus actions (70). Chloroquine has been used in the treatment of malaria and subsequently, autoimmune disorders including rheumatoid arthritis and systemic lupus erythematosus as well as in oncology and paediatric inflammatory disease. It has been reported that chloroquine has been associated with antiviral effects, such as inhibiting pH-dependent steps of the replication of flaviviruses, retroviruses, and coronaviruses (80), for example, on corona infection of primate cells (81). One mechanism of action by which chloroquine may be associated with the observed antiviral effects may be as a result from a possible interference with the terminal glycosylation of ACE2 (81-83). More recently, the Nsp3b or the E-channel (73) have been identified as potential chloroquine targets. It has also been reported that chloroquine has immuno-modulatory effects, suppressing the production/release of tumour necrosis factor and interleukin 6, which mediate the inflammatory complications of several viral diseases (80). Efficacy against malaria parasites has also been associated with a combination of chloroquine and the antiretroviral protease inhibitor indinavir (87). In some embodiments, the composition, such as an ophthalmic, an inhalation, or a topical composition, as disclosed herein, and the methods of using the same, may include, but are not limited to, chloroquine, chlorpromazine, loperamide, indinavir, and/or lopinavir. In some embodiments, the composition is an ophthalmic composition. In some embodiments, the composition is an inhalation composition. In some embodiments, the composition is a topical composition. In some embodiments, the ophthalmic composition, the inhalation composition, or the topical composition, is suitable for oral administration.

Recently, hydroxychloroquine, a derivative of chloroquine, was found to be more potent than chloroquine at inhibiting SARS-CoV-2 in vitro (84). Hydroxychloroquine has a reduced toxicity profile, relative to chloroquine (85). More recently, early reports of a study of non-randomised, unblinded study in Marseille showed a strong reduction in viral load with oral administration of hydroxychloroquine (86). After 6 days, the percentage of patients testing positive for COVID-19 who received 600 mg/day hydroxychloroquine fell to 25% versus 90% for those who did not receive the treatment. Furthermore, comparing untreated patients, those who received 600 mg/day hydroxychloroquine and those given 600 mg/day hydroxychloroquine plus orally administered azithromycin (500 mg on day 1, followed by 250 mg/day for the next four days), early reports indicate achieving a further reduction in the number of positive cases to ~6%. In some embodiments, the composition, such as an ophthalmic, an inhalation, or a topical composition, as disclosed herein, and the methods of using the same, may include hydroxychloroquine or a pharmaceutically acceptable salt thereof. In some embodiments, the composition disclosed herein, and the methods of using the same, may include hydroxychloroquine or a pharmaceutically acceptable salt thereof and azithromycin. In some embodiments, the composition is an ophthalmic composition. In some embodiments, the composition is an inhalation composition. In some embodiments, the composition is a topical composition. In some embodiments, the ophthalmic composition, the inhalation composition, or the topical composition, is suitable for oral administration.

While systemic administration chloroquine and its derivatives, for example resulting from oral administration, appear to be associated with relatively minor side effects in the short term, exacerbation of ECG QT interval prolongation has been reported (88). Systemic administration chloroquine and its derivatives have also been associated with irreversible visual loss due to retinal toxicity, and was traditionally considered a late manifestation particularly seen with high and prolonged dosage. However, a recent review points out that hydroxychloroquine retinal toxicity as a result of systemic administration is far more common than previously considered (87), and with modern imaging techniques (89), is evident well before functional visual loss occurs. Prevalence as high as 7.5% (90) has been reported and is highly dependent on daily dose by weight with a lower risk with doses of ≤5 mg/kg real weight/day.

Of concern is that early-onset (~ 2 months) of chloroquine-associated macular toxicity has been reported (91) in a case where the ideal systemic dosage of chloroquine was exceeded for one month. Predisposing factors to retinal toxicity include a genetic factor (92) - polymorphisms in the cytochrome P450 gene may affect blood concentrations (93). It has been observed that the pattern of retinal disease appears to be different in Caucasian verses Asian patients (97). For example, in Asian patients, early toxicity is pericentral with an extramacular pattern that could be missed by the usual 10° C. visual field screening (97). For example, in Asian patients, 30° visual field testing and SD-OCT, looking for ellipsoid disruption >8° C. from the fovea was recommended (97).

Some insight into the mechanisms of action of action of chloroquine may provide some understanding of the pathways involved in retinal toxicity. For example, chloroquine’s observed anticancer activity when systemically co-administered with zinc has been explained as being a result of chloroquine acting as a zinc ionophore (98). Zinc has also been reported to inhibit coronavirus polymerase activity (99), by blocking virus replication via inhibition of RNA-dependent RNA polymerase (sometimes referred to as replicase) and been reported to block replication of Hepatitis E virus by inhibiting the activity of viral RNA-dependent RNApolymerase (100). These observations may have been the rationale for a proposed systemic co-administration of chloroquine with zinc to treat patients infected with COVID-19 (101), although this intervention has yet to be formally reported (102). Chloroquine (and tamoxifen) have been associated with inducing retinal toxicity, which is believed to be related to their ability to induce a profound decrease in the activity of lysosomal enzymes in cultured retinal pigment epithelial cells (103). It has also been reported that chloroquine (and tamoxifen) may adversely affect the phagocytosis of the retinal outer segments, thereby providing another mechanism by which these compounds may induce retinal toxicity (103). In another retinal pigment epithelial cell model (ARPE-19), it has been reported that chloroquine-induced vacuole formation and cell death, but co-treatment with the zinc ionophore, clioquinol, attenuated the chloroquine-associated toxicity in a zinc-dependent manner, by reversing autophagy arrest (104). It has also been reported that zinc supplementation, used to retard progression of macular degeneration, may work via improving mitochondrial function and preventing lysosome rupture in retinal pigment epithelial cells (105). In some embodiments, the addition of zinc to an anti-coronavirus chloroquine-based or hydroxychloroquine-based treatment regime may simultaneously increase its efficacy and reduce the risk of retinopathy.

Chloroquine, zinc, and ACE inhibitors, have all been used topically in the eye. For example, used as 0.03% chloroquine phosphate eye drops, chloroquine has been reported as being efficacious in managing dry eye syndrome in humans (106,107). For example, ACE inhibitors have been used topically in animal models of glaucoma (108-110), including agents such as Telmisartan, which is a long acting agent having a mean half-life of 24 hours (111). For example, zinc (as zinc ion) has traditionally been used in astringent eye drops or as an excipient, and sources of zinc (as zinc ion) include zinc sulphate (0.25%) (112-114) and zinc chloride (0.005 - 0.015 mmol/L) (115). Chloroquine and ACE2 inhibitors may act at different parts of the ACE2 receptor, thus the combination may provide a synergistic effect, or may permit a reduction in dosage of one or both agents, thereby reducing the risk of potential side effects from either agent.

Systemic co-administration of hydoxychloroquine with azithromycin to coronavirus infected patients has been recently reported (118). However, systemic dosages of hydoxychloroquine (or chloroquine), as discussed above, have been associated with certain toxicities, particularly if used at elevated doses or for long periods of time. Azithrmomycin has previously been safely used topically in the human eye as a 1-1.5% solution to treat ocular infections (119, 120).

In some embodiments, the composition disclosed herein, and the methods of using the same, comprises, or further comprises, ribavirin, interferon alfa-2b (121, 122), or a statin (73, 123, 124), wherein the composition is a topical preparation.

In some embodiments, the composition, such as an ophthalmic, an inhalation, or a topical composition, as disclosed herein, and the methods of using the same, comprises an effective amount of hydroxychloroquine or chloroquine, or a pharmaceutically acceptable salt thereof, such as an effective amount that is suitable for local administration to an eye, a nose, a face, and/or an oral cavity of a subject, such as at an effective amount that is less than a systemically effective amount of the hydroxychloroquine or chloroquine, respectively, and a pharmaceutically acceptable excipient, carrier or diluent. In some embodiments, the composition disclosed herein, may be effective before or after exposure to a virus, such as a virus that enters or infects a subject via association with or binding to an ACE2 receptor in the subject, such as a coronavirus, for example, COVID-19, and so may be used prophylactically and/or therapeutically, according to the methods disclosed herein. In some embodiments, the composition, such as an ophthalmic, an inhalation, or a topical composition, as disclosed herein, and the methods of using the same, comprises (1) an effective amount of a source of zinc ion and (2) an effective amount of hydroxychloroquine or chloroquine, or a pharmaceutically acceptable salt thereof, such as an effective amount that is suitable for local administration to an eye, a nose, a face, and/or an oral cavity of a subject, such as at an effective amount that is less than a systemically effective amount of the hydroxychloroquine or chloroquine, respectively, and a pharmaceutically acceptable excipient, carrier or diluent. In some embodiments, said composition, and methods of using the same, further comprises an effective amount of a vasoconstrictor. In some embodiments, said composition, and methods of using the same, further comprises an effective amount of iota-carrageenan or a pharmaceutically acceptable salt thereof. In some embodiments, the composition, such as an ophthalmic, an inhalation, or a topical composition, as disclosed herein, and the methods of using the same, comprises (1) an effective amount of a source of zinc ion, (2) an effective amount of azithromycin, and (3) an effective amount of hydroxychloroquine or chloroquine, or a pharmaceutically acceptable salt thereof, such as an effective amount that is suitable for local administration to an eye, a nose, a face, and/or an oral cavity of a subject, such as at an effective amount that is less than a systemically effective amount of the hydroxychloroquine or chloroquine, respectively, and a pharmaceutically acceptable excipient, carrier or diluent. In some embodiments, said composition, and methods of using the same, further comprises an effective amount of a vasoconstrictor. In some embodiments, said composition, and methods of using the same, further comprises an effective amount of iota-carrageenan or a pharmaceutically acceptable salt thereof. In some embodiments, the composition, such as an ophthalmic, an inhalation, or a topical composition, as disclosed herein, and the methods of using the same, comprises (1) an effective amount of a source of zinc ion, (2) an effective amount of azithromycin, (3) an effective amount of an ACE2 inhibitor, and (4) an effective amount of hydroxychloroquine or chloroquine, or a pharmaceutically acceptable salt thereof, such as an effective amount that is suitable for local administration to an eye, a nose, a face, and/or an oral cavity of a subject, such as at an effective amount that is less than a systemically effective amount of the hydroxychloroquine or chloroquine, respectively, and a pharmaceutically acceptable excipient, carrier or diluent. In some embodiments, said composition, and methods of using the same, further comprises an effective amount of a vasoconstrictor. In some embodiments, said composition, and methods of using the same, further comprises an effective amount of iota-carrageenan or a pharmaceutically acceptable salt thereof. In some embodiments, the effective amount of the hydroxychloroquine or the chloroquine, or pharmaceutically acceptable salt thereof, that is suitable for local administration to the eye, nose, face, and/or oral cavity, of the subject, is less than a systemically effective amount of said systemically effective amount of said hydroxychloroquine or said chloroquine, respectively, for example, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said systemically effective amount of said hydroxychloroquine or said chloroquine, respectively. In some embodiments, the composition, such as an ophthalmic, an inhalation, or a topical composition, as disclosed herein, and the methods of using the same, comprises the effective amount of the hydroxychloroquine, or pharmaceutically acceptable salt thereof, that is less than the systemically effective amount of said hydroxychloroquine. In some embodiments, the composition, such as an ophthalmic, an inhalation, or a topical composition, as disclosed herein, and the methods of using the same, comprises the effective amount of the chloroquine, or pharmaceutically acceptable salt thereof, that is less than the systemically effective amount of said chloroquine. In some embodiments, the composition, such as an ophthalmic, an inhalation, or a topical composition, as disclosed herein, and the methods of using the same, comprises the effective amount of the source of zinc ion, wherein the effective amount is suitable for local administration to the eye, nose, face, and/or oral cavity, of a subject. In some embodiments, the effective amount of the source of zinc ion is less than a systemically effective amount of said systemically effective amount of said source of zinc ion, for example, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said systemically effective amount of said source of zinc ion. In some embodiments, the source of zinc ion is a pharmaceutically acceptable salt. In some embodiments, the source of zinc ion is an ophthalmically acceptable salt. In some embodiments, the source of zinc ion is zinc sulfate or zinc chloride. In some embodiments, the composition, such as an ophthalmic, an inhalation, or a topical composition, as disclosed herein, and the methods of using the same, comprises the effective amount of the azithromycin, wherein the effective amount is suitable for local administration to the eye, nose, face, and/or oral cavity, of a subject. In some embodiments, the effective amount of the azithromycin is less than a systemically effective amount of said systemically effective amount of said azithromycin, for example, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said systemically effective amount of said azithromycin. In some embodiments, the composition, such as an ophthalmic, an inhalation, or a topical composition, as disclosed herein, and the methods of using the same, comprises the effective amount of the ACE2 inhibitor, wherein the effective amount is suitable for local administration to the eye, nose, face, and/or oral cavity, of a subject. In some embodiments, the effective amount of the ACE2 inhibitor is less than a systemically effective amount of said systemically effective amount of said ACE2 inhibitor, for example, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said systemically effective amount of said ACE2 inhibitor. In some embodiments, the composition, such as an ophthalmic, an inhalation, or a topical composition, as disclosed herein, and the methods of using the same, comprises the effective amount of the vasoconstrictor, wherein the effective amount is suitable for local administration to the eye, nose, face, and/or oral cavity, of a subject. In some embodiments, the effective amount of the vasoconstrictor is less than a systemically effective amount of said systemically effective amount of said vasoconstrictor, for example, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said systemically effective amount of said vasoconstrictor. In some embodiments, the composition is an ophthalmic composition. In some embodiments, the composition is an inhalation composition. In some embodiments, the composition is a topical composition. In some embodiments, the ophthalmic composition, the inhalation composition, or the topical composition, is suitable for oral administration. For example, in some embodiments, the composition is an aqueous composition. For example, in some embodiments, the composition is a solution, an eye drop composition, a liquid spray, an aerosol, a gel, or a lotion.

In some embodiments, the composition, such as an ophthalmic, an inhalation, or a topical composition, as disclosed herein, and the methods of using the same, comprises an effective amount of iota-carrageenan or a pharmaceutically acceptable salt thereof, and a pharmaceutically acceptable excipient, carrier or diluent. Iota-carrageenan has been reported as being used in compositions suitable for inhalation use, such as nasal sprays, to address nasal congestion, for example (131, 132). In some embodiments, the composition disclosed herein, may be effective before or after exposure to a virus, such as a virus that enters or infects a subject via association with or binding to an ACE2 receptor in the subject, such as a coronavirus, for example, COVID-19, and so may be used prophylactically and/or therapeutically, according to the methods disclosed herein. In some embodiments, the composition, such as an ophthalmic, an inhalation, or a topical composition, as disclosed herein, and the methods of using the same, comprises: i) an effective amount of iotacarrageenan or a pharmaceutically acceptable salt thereof; ii) an effective amount of hydroxychloroquine or chloroquine, or a pharmaceutically acceptable salt thereof; and iii) a pharmaceutically acceptable excipient, carrier or diluent. In some embodiments, the composition, such as an ophthalmic, an inhalation, or a topical composition, as disclosed herein, and the methods of using the same, comprises: i) an effective amount of iotacarrageenan or a pharmaceutically acceptable salt thereof; ii) an effective amount of hydroxychloroquine or chloroquine, or a pharmaceutically acceptable salt thereof; iii) an effective amount of azithromycin; and iv) a pharmaceutically acceptable excipient, carrier or diluent. In some embodiments, the composition, such as an ophthalmic, an inhalation, or a topical composition, as disclosed herein, and the methods of using the same, comprises: i) an effective amount of iota-carrageenan or a pharmaceutically acceptable salt thereof; ii) an effective amount of hydroxychloroquine or chloroquine, or a pharmaceutically acceptable salt thereof; iii) an effective amount of azithromycin; iv) an effective amount of a source of zinc ion; and v) a pharmaceutically acceptable excipient, carrier or diluent. In some embodiments, the effective amount of the iota-carrageenan or pharmaceutically acceptable salt thereof present in the composition, such as the ophthalmic, the inhalation, or the topical composition, as disclosed herein, or in the administered composition according to the methods using the same, is an effective amount that is suitable for local administration to an eye, a nose, a face, and/or an oral cavity of a subject, such as at an effective amount that is less than a systemically effective amount of the iota-carrageenan. In some embodiments, the effective amount of the iota-carrageenan or pharmaceutically acceptable salt thereof that is suitable for local administration to the eye, nose, face, and/or oral cavity, of the subject, is less than a systemically effective amount of said systemically effective amount of said iota-carrageenan, for example, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said systemically effective amount of said iota-carrageenan. In some embodiments, the effective amount of the hydroxychloroquine or chloroquine, or pharmaceutically acceptable salt thereof, respectively, the effective amount of the azithromycin, and/or the effective amount of the source of zinc ion, present in the composition, such as the ophthalmic, the inhalation, or the topical composition, as disclosed herein, or in the administered composition according to the methods using the same, is an effective amount that is suitable for local administration to an eye, a nose, a face, and/or an oral cavity of a subject, such as at an effective amount that is less than a systemically effective amount of the hydroxychloroquine or chloroquine, respectively, the azithromycin, and/or the source of zinc ion, respectively. In some embodiments, the administered composition further comprises an effective amount of an ACE2 inhibitor. In some embodiments, the administered composition further comprises an effective amount of a vasoconstrictor. In some embodiments, the effective amount of the hydroxychloroquine or chloroquine, or pharmaceutically acceptable salt thereof, respectively, the effective amount of the azithromycin, the effective amount of the source of zinc ion, the effective amount of the ACE2 inhibitor, and/or the effective amount of the vasoconstrictor, is less than a systemically effective amount of said systemically effective amount of said hydroxychloroquine or chloroquine, respectively, said azithromycin, said source of zinc ion, said ACE2 inhibitor, and/or said vasoconstrictor, for example, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said systemically effective amount of said hydroxychloroquine or chloroquine, respectively, said azithromycin, said source of zinc ion, said ACE2 inhibitor, and/or said vasoconstrictor, respectively. In some embodiments, the composition is an ophthalmic composition. In some embodiments, the composition is an inhalation composition. In some embodiments, the composition is a topical composition. In some embodiments, the ophthalmic composition, the inhalation composition, or the topical composition, is suitable for oral administration. For example, in some embodiments, the composition is an aqueous composition. For example, in some embodiments, the composition is a solution, an eye drop composition, a liquid spray, an aerosol, a gel, or a lotion.

In some embodiments, the effective amount of the source of zinc ion present in the composition, such as the ophthalmic, the inhalation, or the topical composition, as disclosed herein, or in the administered composition according to the methods using the same, is an effective amount that is suitable for local administration to an eye, a nose, a face, and/or an oral cavity of a subject, such as at an effective amount that is less than a systemically effective amount of the source of zinc ion. In some embodiments, the composition, such as an ophthalmic, an inhalation, or a topical composition, as disclosed herein, and the methods of using the same, comprises the source of zinc ion in said composition at a concentration in the range of between about 0.05-0.50 wt%. For example, in some embodiments, the source of zinc ion is present in said composition at a concentration of about 0.05 wt.%, about 0.10 wt.%, about 0.15 wt.%, about 0.20 wt.%, about 0.25 wt.%, about 0.30 wt.%, about 0.35 wt.%, about 0.40 wt.%, about 0.45 wt.%, or about 0.50 wt.%. In some embodiments, the source of zinc ion is present in said composition at a concentration in the range of between about 1-30 uM. For example, in some embodiments, the source of zinc ion is present in said composition at a concentration of about 1 uM, about 2 uM, about 5 uM, about 10 uM, about 15 uM, about 20 uM, about 25 uM, or about 30 uM. In some embodiments, the source of zinc ion is zinc sulfate or zinc chloride. In some embodiments, the source of zinc ion is zinc sulfate. In some embodiments, the source of zinc ion is zinc chloride.

In some embodiments, the effective amount of the azithromycin present in the composition, such as the ophthalmic, the inhalation, or the topical composition, as disclosed herein, or in the administered composition according to the methods using the same, is an effective amount that is suitable for local administration to an eye, a nose, a face, and/or an oral cavity of a subject, such as at an effective amount that is less than a systemically effective amount of the azithromycin. In some embodiments, the composition, such as an ophthalmic, an inhalation, or a topical composition, as disclosed herein, and the methods of using the same, comprises the azithromycin in said composition at a concentration in the range of between about 0.5-3 wt%. For example, in some embodiments, the azithromycin is present in said composition at a concentration of about 0.5 wt.%, about 1.0 wt.%, about 1.5 wt.%, about 2.0 wt.%, about 2.5 wt.%, or about 3.0 wt.%. In some embodiments, the azithromycin is present in said composition at a concentration in the range of between about 1-30 uM. For example, in some embodiments, the azithromycin is present in said composition at a concentration of about 1 uM, about 2 uM, about 5 uM, about 10 uM, about 15 uM, about 20 uM, about 25 uM, or about 30 uM.

In some embodiments, the effective amount of the hydroxychloroquine or pharmaceutically acceptable salt thereof present in the composition, such as the ophthalmic, the inhalation, or the topical composition, as disclosed herein, or in the administered composition according to the methods using the same, is an effective amount that is suitable for local administration to an eye, a nose, a face, and/or an oral cavity of a subject, such as at an effective amount that is less than a systemically effective amount of the hydroxychloroquine. In some embodiments, the composition, such as an ophthalmic, an inhalation, or a topical composition, as disclosed herein, and the methods of using the same, comprises the hydroxychloroquine or pharmaceutically acceptable salt thereof in said composition at a concentration in the range of between about 0.001-0.10 wt%. For example, in some embodiments, the hydroxychloroquine or pharmaceutically acceptable salt thereof is present in said composition at a concentration of about 0.001 wt.%, about 0.005 wt.%, about 0.01 wt.%, about 0.02 wt.%, about 0.03 wt.%, about 0.04 wt.%, about 0.05 wt.%, about 0.06 wt.%, about 0.07 wt.%, about 0.08 wt.%, about 0.09 wt.%, or about 0.10 wt.%. In some embodiments, the hydroxychloroquine or pharmaceutically acceptable salt thereof is present in said composition at a concentration in the range of between about 1-30 uM. For example, in some embodiments, the hydroxychloroquine or pharmaceutically acceptable salt thereof is present in said composition at a concentration of about 1 uM, about 2 uM, about 5 uM, about 10 uM, about 15 uM, about 20 uM, about 25 uM, or about 30 uM. In some embodiments, the hydroxychloroquine or pharmaceutically acceptable salt thereof is hydroxychloroquine phosphate or hydroxychloroquine sulfate.

In some embodiments, the effective amount of the chloroquine or pharmaceutically acceptable salt thereof present in the composition, such as the ophthalmic, the inhalation, or the topical composition, as disclosed herein, or in the administered composition according to the methods using the same, is an effective amount that is suitable for local administration to an eye, a nose, a face, and/or an oral cavity of a subject, such as at an effective amount that is less than a systemically effective amount of the chloroquine. In some embodiments, the composition, such as an ophthalmic, an inhalation, or a topical composition, as disclosed herein, and the methods of using the same, comprises the chloroquine or pharmaceutically acceptable salt thereof in said composition at a concentration in the range of between about 0.001-0.10 wt%. For example, in some embodiments, the chloroquine or pharmaceutically acceptable salt thereof is present in said composition at a concentration of about 0.001 wt.%, about 0.005 wt.%, about 0.01 wt.%, about 0.02 wt.%, about 0.03 wt.%, about 0.04 wt.%, about 0.05 wt.%, about 0.06 wt.%, about 0.07 wt.%, about 0.08 wt.%, about 0.09 wt.%, or about 0.10 wt.%. In some embodiments, the chloroquine or pharmaceutically acceptable salt thereof is present in said composition at a concentration in the range of between about 1-30 uM. For example, in some embodiments, the chloroquine or pharmaceutically acceptable salt thereof is present in said composition at a concentration of about 1 uM, about 2 uM, about 5 uM, about 10 uM, about 15 uM, about 20 uM, about 25 uM, or about 30 uM. In some embodiments, the chloroquine or pharmaceutically acceptable salt thereof is chloroquine phosphate. In some embodiments, the chloroquine or pharmaceutically acceptable salt thereof is chloroquine sulfate.

In some embodiments, the effective amount of the iota-carrageenan or pharmaceutically acceptable salt thereof present in the composition, such as the ophthalmic, the inhalation, or the topical composition, as disclosed herein, or in the administered composition according to the methods using the same, is an effective amount that is suitable for local administration to an eye, a nose, a face, and/or an oral cavity of a subject, such as at an effective amount that is less than a systemically effective amount of said iota-carrageenan. In some embodiments, the composition, such as an ophthalmic, an inhalation, or a topical composition, as disclosed herein, and the methods of using the same, comprises the iotacarrageenan or pharmaceutically acceptable salt thereof in said composition at a concentration in the range of about 1-1.5 mg/mL, such as at a concentration of about 1.2 mg/mL.

In some embodiments, said composition, and methods of using the same, further comprises an effective amount of a vasoconstrictor. A vasoconstrictor may typically be used to reduce redness of the eye (130), however, the vasoconstrictor may be used in the compositions disclosed herein, and methods of using the same, to shut down blood vessels and thereby increase the residence time of other active agents (included in the compositions disclosed herein or administered according to the methods of using the same) on the eye and in the lacrimal drainage system as a result of the vasoconstrictor temporarily limiting the ability of the blood to remove (or carry away) the other active agents, such as the hydroxychloroquine or chloroquine, or a pharmaceutically acceptable salt thereof, the source of zinc ion, the azithromycin, and/or the ACE2 inhibitor. In some embodiments, the vasoconstrictor is a sympathomimetic amine or a sympathomimetic imidazole. In some embodiments, the vasoconstrictor is xylometazoline, naphazoline, tetrahydrozoline, phenylephrine and oxymetazoline, or brimonidine, or a pharmaceutically acceptable salts thereof. In some embodiments, the vasoconstrictor is xylometazoline hydrochloride. In some embodiments, the composition, such as an ophthalmic, an inhalation, or a topical composition, as disclosed herein, and the methods of using the same, comprising (1) an effective amount of a source of zinc ion and (2) an effective amount of hydroxychloroquine or chloroquine, or a pharmaceutically acceptable salt thereof; (1) an effective amount of a source of zinc ion, (2) an effective amount of azithromycin, and (3) an effective amount of hydroxychloroquine or chloroquine, or a pharmaceutically acceptable salt thereof; or (1) an effective amount of a source of zinc ion, (2) an effective amount of azithromycin, (3) an effective amount of an ACE2 inhibitor, and (4) an effective amount of hydroxychloroquine or chloroquine, or a pharmaceutically acceptable salt thereof; may further comprise an effective amount of a vasoconstrictor, such as xylometazoline hydrochloride. For example, an ophthalmic, an inhalation, or a topical composition, as disclosed herein, and the methods of using the same, such as a nasal spray, may further comprise an effective amount of xylometazoline hydrochloride at a concentration of about 0.25-1.0 mg/mL, such as about 0.5 mg/mL.

In some embodiments, the methods of using the ophthalmic, the inhalation, or the topical composition as disclosed herein, comprises administering said composition to a major viral entry point into a subject, such as eyelids, ocular surface, nose, mouth, or combinations thereof. In some embodiments, the composition, the composition, such as an ophthalmic, an inhalation, or a topical composition, as disclosed herein, and the methods of using the same, is prophylactically administered to an eye, an eyelid, an ocular surface, a nose or a mouth of subject, or combinations thereof. For example, in some embodiments, the subject has been exposed to a virus and shows symptoms associated with a healthy individual. In some embodiments, the composition disclosed herein, and the methods of using the same, is therapeutically administered to an eye, an eyelid, an ocular surface, a nose or a mouth of subject, or combinations thereof. For example, in some embodiments, the subject has been exposed to a virus and shows symptoms associated with an individual having a viral infection. In some embodiments, the virus is a virus that enters or infects a subject via association with or binding to an ACE2 receptor in the subject. In some embodiments, the virus is a coronavirus. In some embodiments, the coronavirus is COVID-19. For example, in some embodiments, the subject has been exposed to a virus and shows symptoms associated with an individual having a viral infection, wherein the virus entered or infected the subject via association with or binding to an ACE2 receptor in said subject. For example, in some embodiments, the subject has been exposed to a coronavirus and shows symptoms associated with an individual having a coronaviral infection. For example, in some embodiments, the subject has been exposed to COVID-19 and shows symptoms associated with an individual having a COVID-19 infection.

In some embodiments, provided herein are methods of treating a viralinfection in a subject in need thereof, said method comprising administering a therapeutically effective amount of an ophthalmic, an inhalation, or a topical composition as disclosed herein, to said subject having a viral infection. In some embodiments, provided herein are methods of preventing a viral infection in a subject, said method comprising administering a prophylactically effective amount of an ophthalmic, an inhalation, or a topical composition disclosed herein, to said subject having been exposed to a virus. In some embodiments, the virus is a virus that enters or infects a subject via association with or binding to an ACE2 receptor in the subject. In some embodiments, the virus is a coronavirus. In some embodiments, the coronavirus is COVID-19. In some embodiments, the viral infection is the result from the virus entering or infecting the subject via association with or binding to an ACE2 receptor in the subject. In some embodiments, the viral infection is a coronaviral infection. In some embodiments, the coronaviral infection is a COVID-19 infection.

In some embodiments, the methods provided herein comprise treating a viral infection by administering to a subject exposed to a virus a therapeutically effective amount of an ophthalmic, an inhalation, or a topical composition as disclosed herein, 1 time, 2 times, 3 times or 4 times per day, for 1 day, 2 days, 3 days, 4 days, 5 days, 6 days, 1 week, 2 weeks, or 3 weeks. In some embodiments, the methods provided herein comprise preventing a viral infection by administering to a subject exposed to a virus a prophylactically effective amount of an ophthalmic, an inhalation, or a topical composition as disclosed herein, 1 time, 2 times, 3 times or 4 times per day, for 1 day, 2 days, 3 days, 4 days, 5 days, 6 days, 1 week, 2 weeks, or 3 weeks. In some embodiments, the virus is a virus that enters or infects a subject via association with or binding to an ACE2 receptor in the subject. In some embodiments, the virus is a coronavirus. In some embodiments, the coronavirus is COVID-19. In some embodiments, the viral infection is the result from the virus entering or infecting the subject via association with or binding to an ACE2 receptor in the subject. In some embodiments, the viral infection is a coronaviral infection. In some embodiments, the coronaviral infection is a COVID-19 infection.

In some embodiments, the benefit of the composition, such as an ophthalmic, an inhalation, or a topical composition, as disclosed herein, and the methods of using the same, is that it reduces the dosage amount (or the effective amount) necessary to achieve efficacy in the treated subject. In some embodiments, the benefit of the composition, such as an ophthalmic, an inhalation, or a topical composition, as disclosed herein, and the methods of using the same, is that it avoids administering elevated or prolonged doses of hydoxychloroquine (or chloroquine), thereby reducing the risk of inducing their respective associated toxicities resulting from systemic administration. For example, in some embodiments, the benefit of the composition, such as an ophthalmic, an inhalation, or a topical composition, as disclosed herein, and the methods of using the same, is that it avoids having to administer the hydroxychloroquine or the chloroquine, or pharmaceutically acceptable salt thereof, at the systemically effective amounts that have been associated with cardiac-type toxicities, such as altering the qT interval, or retinal damage toxicities. In some embodiments, the benefit of the composition, such as an ophthalmic, an inhalation, or a topical composition, as disclosed herein, and the methods of using the same, is that it avoids having to pass through the digestive and metabolic systems before reaching the target of the infection, thereby avoiding the necessity of elevating the amount dosed to accommodate for active agent losses due to metabolism.

In some embodiments, the benefit of the composition, such as an ophthalmic, an inhalation, or a topical composition, as disclosed herein, and the therapeutic methods of using the same, is that it may be more efficacious, safer, and a more cost-effective way of treating a subject having a viral infection, relative to systemic administration. In some embodiments, the benefit of the composition, such as an ophthalmic, an inhalation, or a topical composition, as disclosed herein, and the prophylactic methods of using the same, is that it may be more efficacious, safer, and a more cost-effective way of avoiding a viral infection of a subject, relative to systemic administration. Administration of the composition, such as an ophthalmic, an inhalation, or a topical composition, as disclosed herein, comprising hydroxychloroquine, a source of zinc ion, and azithromycin, and optionally an ACE2 inhibitor, or comprising chloroquine, a source of zinc ion, and azithromycin, and optionally an ACE2 inhibitor, according to the methods of using the same, such as by administering topically to the eyelids, ocular surface and perhaps nose and mouth of the subject with a solution/eye drops or spray/aerosol of said composition, would block binding of viral to the likely major entry point into the subject, would reduce the risk of systemic side effects associated with hydroxychloroquine or chloroquine, and would conserve drugs that may become scarce, since the dosage required would be much less than for systemic treatment. In some embodiments, if a subject has active viral infection of the respiratory tree, then said composition disclosed herein may be administered in an inhaled form. In some embodiments, the viral infection is a viral infection resulting from entering or infecting a subject via association with or binding to an ACE2 receptor in the subject. In some embodiments, the viral infection is a coronavirus infection. In some embodiments, the viral infection is a COVID-19 infection.

In some embodiments, the benefit of the composition, such as an ophthalmic, an inhalation, or a topical composition, as disclosed herein, and the methods of using the same, is that every ACE2 receptor from the outer cornea, through the lacrimal drainage system to the respiratory and gastrointestinal systems as well as both nasal and oral cavities, may be directly accessible. For example, if the eye and its adnexae are the main entry site, then eye drops may be used according to the methods disclosed herein. Factors that may make a subject susceptible to coronavirus attack and invasion may also be used to provide a treatment or preventative protection, that is safe, extensive, convenient and at low cost.

The compositions disclosed herein can be formulated into suitable pharmaceutical preparations such as solutions, suspensions, powders, in sterile solutions or suspensions for ophthalmic administration, as well as dry powder inhalers, and for topical administration.

The compositions can be formulated for single dosage administration. Pharmaceutical carriers or vehicles suitable for the ophthalmic, the inhalation, or the topical composition, as disclosed herein, include any such carriers known to those skilled in the art to be suitable for the particular mode of administration.

The ophthalmic, the inhalation, or the topical composition, as disclosed herein, may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to ophthalmically, by inhalation, orally, topically and locally. The compositions may be in liquid, semi-liquid or solid form and any be formulated in a manner suitable for each route of administration.

In some embodiments, methods provided herein include obtaining a sample from the subject, for example to determine if the subject has a viral infection or a viral load. The sample used in the methods provided herein includes body fluids from a subject. Non-limiting examples of body fluids include saliva, blood (e.g., peripheral whole blood, peripheral blood), blood plasma, amniotic fluid, aqueous humor, bile, lymph, menses, serum, and urine. In some embodiments, the sample is collected from a swab, such as a nasopharyngeal swab. In some embodiments, more than one sample from a subject can be obtained. In some embodiments, the viral infection is a viral infection resulting from entering or infecting a subject via association with or binding to an ACE2 receptor in the subject. In some embodiments, the viral infection is a coronavirus infection. In some embodiments, the viral infection is a COVID-19 infection.

It is understood that subheadings throughout this document do not limit the subject matter discussed to only those sections, but apply, and are contemplated to apply, to each embodiment disclosed in the instant application.

It is understood that modifications which do not substantially affect the activity of the various embodiments of this invention are also provided within the definition of the invention provided herein. Accordingly, the following examples are intended to illustrate but not limit the present invention. All of the references cited to herein are incorporated by reference in their entireties.

### Exemplary Embodiments

A1. In an embodiment, a composition, comprising:
i) an effective amount of hydroxychloroquine or chloroquine, or a pharmaceutically acceptable salt thereof;
ii) an effective amount of a source of zinc ion; and
iii) a pharmaceutically acceptable excipient, carrier or diluent; wherein the composition is an ophthalmic, an inhalation, or a topical composition.

A2. In an embodiment, a composition, comprising:
i) an effective amount of hydroxychloroquine or chloroquine, or a pharmaceutically acceptable salt thereof;
ii) an effective amount of azithromycin; and
iii) a pharmaceutically acceptable excipient, carrier or diluent; wherein the composition is an ophthalmic, an inhalation, or a topical composition.

A3. In an embodiment, a composition, comprising:
i) an effective amount of hydroxychloroquine or chloroquine, or a pharmaceutically acceptable salt thereof;
ii) an effective amount of a source of zinc ion;
iii) an effective amount of azithromycin; and
iv) a pharmaceutically acceptable excipient, carrier or diluent; wherein the composition is an ophthalmic, an inhalation, or a topical composition.

A4. In an embodiment, a composition, comprising:
i) an effective amount of iota-carrageenan or a pharmaceutically acceptable salt thereof; and
ii) a pharmaceutically acceptable excipient, carrier or diluent; wherein the composition is an ophthalmic, an inhalation, or a topical composition.

A5. In an embodiment, a composition, comprising:
i) an effective amount of iota-carrageenan or a pharmaceutically acceptable salt thereof;
ii) an effective amount of hydroxychloroquine or chloroquine, or a pharmaceutically acceptable salt thereof; and
iii) a pharmaceutically acceptable excipient, carrier or diluent; wherein the composition is an ophthalmic, an inhalation, or a topical composition.

A6. In an embodiment, a composition, comprising:
i) an effective amount of iota-carrageenan or a pharmaceutically acceptable salt thereof;
ii) an effective amount of hydroxychloroquine or chloroquine, or a pharmaceutically acceptable salt thereof;
iii) an effective amount of azithromycin; and
iv) a pharmaceutically acceptable excipient, carrier or diluent; wherein the composition is an ophthalmic, an inhalation, or a topical composition.

A7. In an embodiment, a composition, comprising:
i) an effective amount of iota-carrageenan or a pharmaceutically acceptable salt thereof;
ii) an effective amount of hydroxychloroquine or chloroquine, or a pharmaceutically acceptable salt thereof;
iii) an effective amount of azithromycin;
iv) an effective amount of a source of zinc ion; and
v) a pharmaceutically acceptable excipient, carrier or diluent; wherein the composition is an ophthalmic, an inhalation, or a topical composition.

In certain embodiments, one or more than one (including for instance all) of the following further embodiments may comprise each of the other embodiments or parts thereof.

A8. The composition of embodiment A1, wherein the composition further comprises an effective amount of iota-carrageenan or a pharmaceutically acceptable salt thereof.

A9. The composition of any one of embodiments A1-A8, wherein the composition further comprises an effective amount of an ACE2 inhibitor.

A10. The composition of any one of embodiments A1-A9, wherein the composition further comprises an effective amount of a vasoconstrictor.

A11. The composition of any one of embodiments A1-A3 or embodiments A5-A10, wherein the effective amount of the hydroxychloroquine or the chloroquine, or pharmaceutically acceptable salt thereof, is an amount suitable for local administration to an eye, a nose, a face, and/or an oral cavity of a subject.

A12. The composition of any one of embodiments A1-A3 or embodiments A5-A11, wherein the effective amount of the hydroxychloroquine or the chloroquine, or pharmaceutically acceptable salt thereof, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said hydroxychloroquine or said chloroquine, respectively.

A13. The composition of any one of embodiment A1, embodiment A3, or embodiments A7-A12, wherein the effective amount of the source of zinc ion is an amount suitable for local administration to an eye, a nose, a face, and/or an oral cavity of a subject.

A14. The composition of any one of embodiment A1, embodiment A3, or embodiments A7-A13, wherein the effective amount of the source of zinc ion is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said source of zinc ion.

A15. The composition of any one of embodiment A1, embodiment A3, or embodiments A7-A14, wherein the effective amount of the source of zinc ion is a prophylactically effective amount of said source of zinc ion.

A16. The composition of any one of embodiment A1, embodiment A3, or embodiments A7-A14, wherein the effective amount of the source of zinc ion is a therapeutically effective amount of said source of zinc ion.

A17. The composition of any one of embodiment A1, embodiment A3, or embodiments A7-A16, wherein the source of zinc ion is a pharmaceutically acceptable salt.

A18. The composition of any one of embodiment A1, embodiment A3, or embodiments A7-A17, wherein the source of zinc ion is an ophthalmically acceptable salt.

A19. The composition of any one of embodiment A1, embodiment A3, or embodiments A7-A18, wherein the source of zinc ion is present in said composition at a concentration in the range of between about 0.05-0.50 wt%.

A20. The composition of any one of embodiment A1, embodiment A3, or embodiments A7-A19, wherein the source of zinc ion is present in said composition at a concentration of about 0.05 wt.%, about 0.10 wt.%, about 0.15 wt.%, about 0.20 wt.%, about 0.25 wt.%, about 0.30 wt.%, about 0.35 wt.%, about 0.40 wt.%, about 0.45 wt.%, or about 0.50 wt.%.

A21. The composition of any one of embodiment A1, embodiment A3, or embodiments A7-A20, wherein the source of zinc ion is present in said composition at a concentration in the range of between about 1-30 uM.

A22. The composition of any one of embodiment A1, embodiment A3, or embodiments A7-A21, wherein the source of zinc ion is present in said composition at a concentration of about 1 uM, about 2 uM, about 5 uM, about 10 uM, about 15 uM, about 20 uM, about 25 uM, or about 30 uM.

A23. The composition of any one of embodiment A1, embodiment A3, or embodiments A7-A22, wherein the source of zinc ion is zinc sulfate or zinc chloride.

A24. The composition of any one of embodiment A1, embodiment A3, or embodiments A7-A23, wherein the source of zinc ion is zinc sulfate.

A25. The composition of any one of embodiment A1, embodiment A3, or embodiments A7-A24, wherein the source of zinc ion is zinc chloride.

A26. The composition of any one of embodiments A2-A3 or embodiments A6-A25, wherein the effective amount of the azithromycin is an amount suitable for local administration to an eye, a nose, a face, and/or an oral cavity of a subject.

A27. The composition of any one of embodiments A2-A3 or embodiments A6-A26, wherein the effective amount of the azithromycin is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said azithromycin.

A28. The composition of any one of embodiments A2-A3 or embodiments A6-A27, wherein the effective amount of the azithromycin is a prophylactically effective amount of said azithromycin.

A29. The composition of any one of embodiments A2-A3 or embodiments A6-A28, wherein the effective amount of the azithromycin is a therapeutically effective amount of said azithromycin.

A30. The composition of any one of embodiments A2-A3 or embodiments A6-A29, wherein the azithromycin is present in said composition at a concentration in the range of between about 0.5-3 wt%.

A31. The composition of any one of embodiments A2-A3 or embodiments A6-A30, wherein the azithromycin is present in said composition at a concentration of about 0.5 wt.%, about 1.0 wt.%, about 1.5 wt.%, about 2.0 wt.%, about 2.5 wt.%, or about 3.0 wt.%.

A32. The composition of any one of embodiments A2-A3 or embodiments A6-A31, wherein the azithromycin is present in said composition at a concentration in the range of between about 1-30 uM.

A33. The composition of any one of embodiments A2-A3 or embodiments A6-A32, wherein the azithromycin is present in said composition at a concentration of about 1 uM, about 2 uM, about 5 uM, about 10 uM, about 15 uM, about 20 uM, about 25 uM, or about 30 uM.

A34. The composition of any one of embodiments A4-A3, wherein the effective amount of the iota-carrageenan or pharmaceutically acceptable salt thereof is an amount suitable for local administration to an eye, a nose, a face, and/or an oral cavity of a subject.

A35. The composition of any one of embodiments A4-A34, wherein the effective amount of the iota-carrageenan or pharmaceutically acceptable salt thereof is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said iotacarrageenan or pharmaceutically acceptable salt thereof.

A36. The composition of any one of embodiments A4-A35, wherein the effective amount of the iota-carrageenan or pharmaceutically acceptable salt thereof is a prophylactically effective amount of said iota-carrageenan or pharmaceutically acceptable salt thereof.

A37. The composition of any one of embodiments A4-A35, wherein the effective amount of the iota-carrageenan or pharmaceutically acceptable salt thereof is a therapeutically effective amount of said iota-carrageenan or pharmaceutically acceptable salt thereof.

A38. The composition of any one of embodiments A4-A37, wherein the iotacarrageenan or pharmaceutically acceptable salt thereof is present in said composition at a concentration in the range of between about 1.0-1.5 mg/mL.

A39. The composition of any one of embodiments A4-A38, wherein the iotacarrageenan or pharmaceutically acceptable salt thereof is present in said composition at a concentration of about 1.2 mg/mL.

A40. The composition of any one of embodiments A9-A39, wherein the effective amount of the ACE2 inhibitor is an amount suitable for local administration to an eye, a nose, a face, and/or an oral cavity of a subject.

A41. The composition of any one of embodiments A9-A40, wherein the effective amount of the ACE2 inhibitor is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said ACE2 inhibitor.

A42. The composition of any one of embodiments A9-A41, wherein the effective amount of the ACE2 inhibitor is a prophylactically effective amount of said ACE2 inhibitor.

A43. The composition of any one of embodiments A9-A41, wherein the effective amount of the ACE2 inhibitor is a therapeutically effective amount of said ACE2 inhibitor.

A44. The composition of any one of embodiments A9-A43, wherein the ACE2 inhibitor is present in said composition at a concentration in the range of between about 0.01-3 wt%.

A45. The composition of any one of embodiments A9-A44, wherein the ACE2 inhibitor is present in said composition at a concentration of about 0.1 wt.%, 0.5 wt.%, about 1.0 wt.%, about 1.5 wt.%, about 2.0 wt.%, about 2.5 wt.%, or about 3.0 wt.%.

A46. The composition of any one of embodiments A9-A45, wherein the ACE2 inhibitor is MLN-4760 or DX600.

A47. The composition of any one of embodiments A10-A46, wherein the effective amount of the vasoconstrictor is an amount suitable for local administration to an eye, a nose, a face, and/or an oral cavity of a subject.

A48. The composition of any one of embodiments A10-A47, wherein the effective amount of the vasoconstrictor is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said vasoconstrictor.

A49. The composition of any one of embodiments A10-A48, wherein the effective amount of the vasoconstrictor is a prophylactically effective amount of said vasoconstrictor.

A50. The composition of any one of embodiments A10-A48, wherein the effective amount of the vasoconstrictor is a therapeutically effective amount of said vasoconstrictor.

A51. The composition of any one of embodiments A10-A50, wherein the vasoconstrictor is vasoconstrictor is a sympathomimetic amine or a sympathomimetic imidazole.

A52. The composition of any one of embodiments A10-A51, wherein the vasoconstrictor is vasoconstrictor is xylometazoline, naphazoline, tetrahydrozoline, phenylephrine and oxymetazoline, or brimonidine, or a pharmaceutically acceptable salts thereof.

A53. The composition of any one of embodiments A10-A52, wherein the vasoconstrictor is xylometazoline hydrochloride.

A54. The composition of any one of embodiments A10-A53, wherein the xylometazoline hydrochloride is present in said composition at a concentration in the range of between about 0.25-1.0 mg/mL.

A55. The composition of any one of embodiments A10-A54, wherein the xylometazoline hydrochloride is present in said composition at a concentration of about 0.5 mg/mL.

A56. The composition of any one of embodiments A1-A3 or embodiments A5-A55, wherein the composition comprises the hydroxychloroquine or pharmaceutically acceptable salt thereof.

A57. The composition of embodiment A56, wherein the composition comprises the effective amount of the hydroxychloroquine or pharmaceutically acceptable salt thereof is a prophylactically effective amount of said hydroxychloroquine.

A58. The composition of embodiment A56, wherein the composition comprises the effective amount of the hydroxychloroquine or pharmaceutically acceptable salt thereof is a therapeutically effective amount of said hydroxychloroquine.

A59. The composition of any one of embodiments A1-A3 or embodiments A5-A58, wherein the hydroxychloroquine or pharmaceutically acceptable salt thereof is present in said composition at a concentration in the range of between about 0.001-0.10 wt%.

A60. The composition of any one of embodiments A1-A3 or embodiments A5-A59, wherein the hydroxychloroquine or pharmaceutically acceptable salt thereof is present in said composition at a concentration of about 0.001 wt.%, about 0.005 wt.%, about 0.01 wt.%, about 0.02 wt.%, about 0.03 wt.%, about 0.04 wt.%, about 0.05 wt.%, about 0.06 wt.%, about 0.07 wt.%, about 0.08 wt.%, about 0.09 wt.%, or about 0.10 wt.%.

A61. The composition of any one of embodiments A1-A3 or embodiments A5-A60, wherein the hydroxychloroquine or pharmaceutically acceptable salt thereof is present in said composition at a concentration in the range of between about 1-30 uM.

A62. The composition of any one of embodiments A1-A3 or embodiments A5-A61, wherein the hydroxychloroquine or pharmaceutically acceptable salt thereof is present in said composition at a concentration of about 1 uM, about 2 uM, about 5 uM, about 10 uM, about 15 uM, about 20 uM, about 25 uM, or about 30 uM.

A63. The composition of any one of embodiments A1-A3 or embodiments A5-A62, wherein the hydroxychloroquine or pharmaceutically acceptable salt thereof is hydroxychloroquine phosphate or hydroxychloroquine sulfate.

A64. The composition of any one of embodiments A1-A3 or embodiments A5-A55, wherein the composition comprises the chloroquine or pharmaceutically acceptable salt thereof.

A65. The composition of embodiment A64, wherein the composition comprises the effective amount of the chloroquine or pharmaceutically acceptable salt thereof is a prophylactically effective amount of said chloroquine.

A66. The composition of embodiment A64, wherein the composition comprises the effective amount of the chloroquine or pharmaceutically acceptable salt thereof is a therapeutically effective amount of said chloroquine.

A67. The composition of any one of embodiments A1-A3, embodiments A5-A55, or embodiments A64-A66, wherein the chloroquine or pharmaceutically acceptable salt thereof is present in said composition at a concentration in the range of between about 0.001-0.10 wt%.

A68. The composition of any one of embodiments A1-A3, embodiments A5-A55, or embodiments A64-A67, wherein the chloroquine or pharmaceutically acceptable salt thereof is present in said composition at a concentration of about 0.001 wt.%, about 0.005 wt.%, about 0.01 wt.%, about 0.02 wt.%, about 0.03 wt.%, about 0.04 wt.%, about 0.05 wt.%, about 0.06 wt.%, about 0.07 wt.%, about 0.08 wt.%, about 0.09 wt.%, or about 0.10 wt.%.

A69. The composition of any one of embodiments A1-A3, embodiments A5-A55, or embodiments A64-A68, wherein the chloroquine or pharmaceutically acceptable salt thereof is present in said composition at a concentration in the range of between about 1-30 uM.

A70. The composition of any one of embodiments A1-A3, embodiments A5-A55, or embodiments A64-A69, wherein the chloroquine or pharmaceutically acceptable salt thereof is present in said composition at a concentration of about 1 uM, about 2 uM, about 5 uM, about 10 uM, about 15 uM, about 20 uM, about 25 uM, or about 30 uM.

A71. The composition of any one of embodiments A1-A3, embodiments A5-A55, or embodiments A64-A70, wherein the chloroquine or pharmaceutically acceptable salt thereof is chloroquine phosphate.

A72. The composition of any one of embodiments A1-A71, wherein the composition further comprises chlorpromazine, loperamide, indinavir, and/or lopinavir.

A73. The composition of any one of embodiments A1-A72, wherein the composition is a topical composition.

A74. The composition of any one of embodiments A1-A73, wherein the composition is an ophthalmic composition.

A75. The composition of any one of embodiments A1-A74, wherein the composition is an inhalation composition.

A76. The composition of any one of embodiments A1-A75, wherein the composition is suitable for oral administration.

A77. The composition of any one of embodiments A1-A76, wherein the composition is a solution, a spray, an aerosol, a gel, or a lotion.

A78. The composition of any one of embodiments A1-A77, wherein the composition is an aqueous composition.

A79. The composition of any one of embodiments A1-A78, wherein the composition is a sterile solution.

A80. The composition of any one of embodiments A1-A79, wherein the composition is a liquid spray.

A81. The composition of any one of embodiments A1-A80, wherein the composition is an eye drop composition.

A82. A method of treating a subject exposed to a virus, comprising administering to a subject exposed to a virus an effective amount of the composition of any one of embodiments A1-A81, wherein the virus is a virus that enters or infects the subject via association with or binding to an ACE2 receptor in the subject, or the virus is a coronavirus, for example, the virus is a COVID-19 virus.

A83. A method of preventing or treating a viral infection in a subject, comprising administering to a subject exposed to a virus an effective amount of the composition of any one of embodiments A1-A81, wherein the virus is a virus that enters or infects the subject via association with or binding to an ACE2 receptor in the subject, or the virus is a coronavirus, for example, the virus is a COVID-19 virus.

A84. A method of prophylactically treating, comprising administering to a subject prior to exposure to a virus an effective amount of the composition of any one of embodiments A1-A81, wherein the virus is a virus that enters or infects the subject via association with or binding to an ACE2 receptor in the subject, or the virus is a coronavirus, for example, the virus is a COVID-19 virus.

A85. The method of embodiment A84, wherein the subject prior to exposure to a virus does not have symptoms associated with a viral infection, wherein the virus is a virus that enters or infects the subject via association with or binding to an ACE2 receptor in the subject, or the virus is a coronavirus, for example, the virus is a COVID-19 virus.

A86. The method of any one of embodiments A82-A85, wherein the composition is administered topically.

A87. The method of any one of embodiments A82-A86, wherein the composition is administered ophthalmically.

A88. The method of any one of embodiments A82-A87, wherein the composition is administered via inhalation.

A89. The method of any one of embodiments A82-A88, wherein the composition is administered to an ocular surface and/or eyelid of the subject.

A90. The method of any one of embodiments A82-A89, wherein the composition is administered to the subject’s face.

A91. The method of any one of embodiments A82-A90, wherein the composition is administered in a nostril of the subject.

A92. The method of any one of embodiments A82-A91, wherein the administered composition is a topical composition.

A93. The method of embodiment A82, wherein the administered topical composition is suitable for oral administration.

A94. The method of any one of embodiments A82-A93, wherein the administered composition is an ophthalmic composition.

A95. The method of embodiment A94, wherein the administered ophthalmic composition is suitable for oral administration.

A96. The method of any one of embodiments A82-A95, wherein the administered composition is an inhalation composition.

A97. The method of embodiment A96, wherein the inhalation composition is administered via a puffer device suitable for inhalation.

A98. The method of embodiment A96 or embodiment A97, wherein the inhalation composition is administered as a spray or as an aerosol by inhalation via the puffer device.

A99. The method of any one of embodiments A96-A98, wherein the inhalation composition is further administered topically to an ocular surface, an eyelid, in a nostril, or to the face of the subject via spraying the contents within said puffer device.

A100. The method of any one of embodiments A82-A99, wherein the administered inhalation composition is suitable for oral administration.

A101. The method of any one of embodiments A82-A100, wherein the administered composition is further administered to the surface of the subject’s oral cavity.

A102. The method of any one of embodiments A82-A101, wherein the exposed subject does not have symptoms associated with a viral infection, wherein the viral infection is a viral infection resulting from entering or infecting a subject via association with or binding to an ACE2 receptor in the subject, or the viral infection is a coronavirus infection, for example, the viral infection is a COVID-19 infection.

A103. The method of any one of embodiments A82-A102, wherein the method is a preventative treatment of the subject exposed to the virus to avoid contracting a viral infection, wherein the virus is a virus that enters or infects the subject via association with or binding to an ACE2 receptor in the subject, or the virus is a coronavirus, for example, the virus is a COVID-19 virus.

A104. The method of any one of embodiments A82-A103, wherein the method is a prophylactic treatment of the subject exposed to the virus, wherein the virus is a virus that enters or infects the subject via association with or binding to an ACE2 receptor in the subject, or the virus is a coronavirus, for example, the virus is a COVID-19 virus.

A105. The method of any one of embodiments A82-A101, wherein the exposed subject has one or more symptoms associated with a viral infection, wherein the viral infection is a viral infection resulting from entering or infecting a subject via association with or binding to an ACE2 receptor in the subject, or the viral infection is a coronavirus infection, for example, the viral infection is a COVID-19 infection.

A106. The method of any one of embodiments A82-A101 or embodiment A105, wherein the exposed subject has a viral infection, wherein the viral infection is a viral infection resulting from entering or infecting a subject via association with or binding to an ACE2 receptor in the subject, or the viral infection is a coronavirus infection, for example, the viral infection is a COVID-19 infection.

A107. The method of any one of embodiments A82-A101 or embodiments A105-A106, wherein the method is a therapeutic treatment of the subject having the viral infection, wherein the viral infection is a viral infection resulting from entering or infecting a subject via association with or binding to an ACE2 receptor in the subject, or the viral infection is a coronavirus infection, for example, the viral infection is a COVID-19 infection.

A108. The method of any one of embodiments A82-A107, wherein the effective amount of the composition is administered to the subject 1 time, 2 times, 3 times or 4 times per day, for 1 day, 2 days, 3 days, 4 days, 5 days, 6 days, 1 week, 2 weeks, or 3 weeks.

A109. The method of any one of embodiments A82-A108, wherein the amount of the administered composition is a prophylactically effective amount.

A110. The method of any one of embodiments A82-A109, wherein the amount of the administered composition is a therapeutically effective amount.

A111. The method of any one of embodiments A82-A110, wherein the virus is a virus that enters or infects the subject via association with or binding to an ACE2 receptor in the subject.

A112. The method of any one of embodiments A82-A110, wherein the virus is a coronavirus.

A113. The method of any one of embodiments A82-A110, wherein the virus is a COVID-19 virus.

A114. The method of any one of embodiments A82-A113, wherein the viral infection is a viral infection resulting from entering or infecting a subject via association with or binding to an ACE2 receptor in the subject.

A115. The method of any one of embodiments A82-A113, wherein the viral infection is a coronavirus infection.

A116. The method of any one of embodiments A82-A113, wherein the viral infection is a COVID-19 infection.

### REFERENCES

1. Perlman S. Another Decade, Another Coronavirus. N Engl J Med. 2020 Jan 24;10.1056/NEJMe2001126. doi: 10.1056/NEJMe2001126. [Epub ahead of print]. PMID: 31978944.

2. Shen Z, Ning F, Zhou W, He X, Lin C, Chin DP, Zhu Z, Schuchat A. Superspreading SARS events, Beijing, 2003. Emerg Infect Dis. 2004 Feb;10(2):256-60. doi: 10.3201/eid1002.030732. PMID: 15030693; PMCID: PMC3322930.

3. Li Q, Guan X, Wu P, Wang X, Zhou L, Tong Y, Ren R, Leung KSM, Lau EHY, Wong JY, Xing X, Xiang N, Wu Y, Li C, Chen Q, Li D, Liu T, Zhao J, Li M, Tu W, Chen C, Jin L, Yang R, Wang Q, Zhou S, Wang R, Liu H, Luo Y, Liu Y, Shao G, Li H, Tao Z, Yang Y, Deng Z, Liu B, Ma Z, Zhang Y, Shi G, Lam TTY, Wu JTK, Gao GF, Cowling BJ, Yang B, Leung GM, Feng Z. Early Transmission Dynamics in Wuhan, China, of Novel Coronavirus-Infected Pneumonia. N Engl J Med. 2020 Jan 29;10.1056/NEJMoa2001316. doi: 10.1056/NEJMoa2001316. [Epub ahead of print]. PMID: 31995857.

4. Peng X, Xu X, Li Y, Cheng L, Zhou X, Ren B. Transmission routes of 2019-nCoV and controls in dental practice. Int J Oral Sci. 2020 Mar 3;12(1):9. doi: 10.1038/s41368-020-0075-9. PMID: 32127517.

5. Chan WM, Yuen KS, Fan DS, Lam DS, Chan PK, Sung JJ. Tears and conjunctival scrapings for coronavirus in patients with SARS. Br J Ophthalmol. 2004 Jul;88(7):968-9. doi: 10.1136/bjo.2003.039461. PMID: 15205249; PMCID: PMC1772218.

6. Belser JA, Rota PA, Tumpey TM. Ocular tropism of respiratory viruses. Microbiol Mol Biol Rev. 2013 Mar;77(1):144-56. doi: 10.1128/MMBR.00058-12. PMID: 23471620; PMCID: PMC3591987.

7. WHO | Update 27 - One month into the global SARS outbreak: Status of the outbreak and lessons for the immediate future.

8. Tong T, Lai TS. The severe acute respiratory syndrome coronavirus in tears. Br J Ophthalmol. 2005 Mar;89(3):392. doi: 10.1136/bjo.2004.054130. PMID: 15722333; PMCID: PMC1772561.

9. Loon SC, Teoh SC, Oon LL, Se-Thoe SY, Ling AE, Leo YS, Leong HN. The severe acute respiratory syndrome coronavirus in tears. Br J Ophthalmol. 2004 Jul;88(7):861-3. doi: 10.1136/bjo.2003.035931. PMID: 15205225; PMCID: PMC1772213.

10. van der Hoek L, Pyrc K, Jebbink MF, Vermeulen-Oost W, Berkhout RJ, Wolthers KC, Wertheim-van Dillen PM, Kaandorp J, Spaargaren J, Berkhout B. Identification of a new human coronavirus. Nat Med. 2004 Apr;10(4):368-73. doi: 10.1038/nm1024. Epub 2004 Mar 21. PMID: 15034574.

11. Vabret A, Mourez T, Dina J, van der Hoek L, Gouarin S, Petitjean J, Brouard J, Freymuth F. Human coronavirus NL63, France. Emerg Infect Dis. 2005 Aug;11(8):1225-9. doi: 10.3201/eid1108.050110. PMID: 16102311; PMCID: PMC3320486.

12. Xia J, Tong J, Liu M, Shen Y, Guo D. Evaluation of coronavirus in tears and conjunctival secretions of patients with SARS-CoV-2 infection. J Med Virol. 2020 Feb 26. doi: 10.1002/jmv.25725. Epub ahead of print. PMID: 32100876.

13. Guan WJ, Ni ZY, Hu Y, Liang WH, Ou CQ, He JX, Liu L, Shan H, Lei CL, Hui DSC, Du B, Li LJ, Zeng G, Yuen KY, Chen RC, Tang CL, Wang T, Chen PY, Xiang J, Li SY, Wang JL, Liang ZJ, Peng YX, Wei L, Liu Y, Hu YH, Peng P, Wang JM, Liu JY, Chen Z, Li G, Zheng ZJ, Qiu SQ, Luo J, Ye CJ, Zhu SY, Zhong NS; China Medical Treatment Expert Group for Covid-19. Clinical Characteristics of Coronavirus Disease 2019 in China. N Engl J Med. 2020 Feb 28. doi: 10.1056/NEJMoa2002032. Epub ahead of print. PMID: 32109013.

14. Maxcy KF. The transmission of infection through the eye. JAMA. 1919;72:636-639. doi:10.1001/jama.1919.02610090020005.

15. Zaman ML, Doughty MJ, Button NF. The exposed ocular surface and its relationship to spontaneous eyeblink rate in elderly caucasians. Exp Eye Res. 1998 Dec;67(6):681-6. doi: 10.1006/exer.1998.0571. PMID: 9990332.

16. Sotoyama, M., Villanueva, M. B. G., Jonai, H. and Saito, S. (1995). Ocular surface area as an informative index of visual ergonomics. Ind. Health. 33, 43-56.

17. Ehlers N. On the size of the conjunctival sac. Acta Ophthalmol 1965;43:205-210.

18. Watsky MA, Jablonski MM, Edelhauser HF. Comparison of conjunctival and corneal surface areas in rabbit and human. Curr Eye Res. 1988 May;7(5):483-6. doi: 10.3109/02713688809031801. PMID: 3409715.

19. Özer CM, Öz II, Serifoğlu I, Büyükuysal MÇ, Barut Ç. Evaluation of Eyeball and Orbit in Relation to Gender and Age. J Craniofac Surg. 2016 Nov;27(8):e793-e800. doi: 10.1097/SCS.0000000000003133. PMID: 28005828.

20. Kanjani V, Rani A, Kanjani D. Morphometric Analysis of the Orbital Aperture in North Indian Population: A Retrospective Digital Forensic Study. Int J Appl Basic Med Res. 2019 Apr-Jun;9(2):85-88. doi: 10.4103/ijabmr.IJABMR_404_18. PMID: 31041170; PMCID: PMC6477950.

21. Ng A, Evans K, North RV, Purslow C. Migration of Cosmetic Products into the Tear Film. Eye Contact Lens. 2015 Sep;41(5):304-9. doi: 10.1097/ICL.0000000000000124. PMID: 25738987.

22. MacKeen DL, Roth HW, Doane MG, MacKeen PD. Supracutaneous treatment of dry eye patients with calcium carbonate. Adv Exp Med Biol. 1998;438:985-90. doi: 10.1007/978-1-4615-5359-5 _141. PMID: 9634999.

23. Tsubota K, Monden Y, Yagi Y, Goto E, Shimmura S. New treatment of dry eye: the effect of calcium ointment through eyelid skin delivery. Br J Ophthalmol. 1999 Jul;83(7):767-70. doi: 10.1136/bjo.83.7.767. PMID: 10381659; PMCID: PMC1723096.

24. Amador GJ, Mao W, DeMercurio P, Montero C, Clewis J, Alexeev A, Hu DL. Eyelashes divert airflow to protect the eye. J R Soc Interface. 2015 Apr 6;12(105):20141294. doi: 10.1098/rsif.2014.1294. PMID: 25716186; PMCID: PMC4387520.

25. Zou S, Zha J, Xiao J, Chen XD. How eyelashes can protect the eye through inhibiting ocular water evaporation: a chemical engineering perspective. J R Soc Interface. 2019 Oct 31;16(159):20190425. doi: 10.1098/rsif.2019.0425. Epub 2019 Oct 9. PMID: 31594526; PMCID: PMC6833320.

26. Na JI, Kwon OS, Kim BJ, Park WS, Oh JK, Kim KH, Cho KH, Eun HC. Ethnic characteristics of eyelashes: a comparative analysis in Asian and Caucasian females. Br J Dermatol. 2006 Dec;155(6):1170-6. doi: 10.1111/j.1365-2133.2006.07495.x. PMID: 17107385.

27. Verreault D, Moineau S, Duchaine C. Methods for sampling of airborne viruses. Microbiol Mol Biol Rev. 2008 Sep;72(3):413-44. doi: 10.1128/MMBR.00002-08. PMID: 18772283; PMCID: PMC2546863.

28. Tellier R, Li Y, Cowling BJ, Tang JW. Recognition of aerosol transmission of infectious agents: a commentary. BMC Infect Dis. 2019 Jan 31; 19(1): 101. doi: 10.1186/s12879-019-3707-y. PMID: 30704406; PMCID: PMC6357359.

29. Clayton JA. Dry Eye. N Engl J Med. 2018 Jun 7;378(23):2212-2223. doi: 10.1056/NEJMra1407936. PMID: 29874529.

30. Pappas A. Epidermal surface lipids. Dermatoendocrinol. 2009 Mar;1(2):72-6. doi: 10.4161/derm.1.2.7811. PMID: 20224687; PMCID: PMC2835894.

31. Chapman SJ, Walsh A, Jackson SM, Friedmann PS. Lipids, proteins and corneocyte adhesion. Arch Dermatol Res. 1991;283(3):167-73. doi: 10.1007/bf00372057. PMID: 1867479.

32. Butovich IA. Tear film lipids. Exp Eye Res. 2013 Dec;117:4-27. doi: 10.1016/j.exer.2013.05.010. Epub 2013 Jun 12. PMID: 23769846; PMCID: PMC3844095.

33. Rey FA, Lok SM. Common Features of Enveloped Viruses and Implications for Immunogen Design for Next-Generation Vaccines. Cell. 2018 Mar 8;172(6):1319-1334. doi: 10.1016/j.cell.2018.02.054. PMID: 29522750.

34. Choi KS, Aizaki H, Lai MM. Murine coronavirus requires lipid rafts for virus entry and cell-cell fusion but not for virus release. J Virol. 2005 Aug;79(15):9862-71. doi: 10.1128/JVI.79.15.9862-9871.2005. PMID: 16014947; PMCID: PMC1181594.

35. Glende J, Schwegmann-Wessels C, Al-Falah M, Pfefferle S, Qu X, Deng H, Drosten C, Naim HY, Herrler G. Importance of cholesterol-rich membrane microdomains in the interaction of the S protein of SARS-coronavirus with the cellular receptor angiotensin-converting enzyme 2. Virology. 2008 Nov 25;381(2):215-21. doi: 10.1016/j.virol.2008.08.026. Epub 2008 Sep 23. PMID: 18814896.

36. Dartt DA, Willcox MD. Complexity of the tear film: importance in homeostasis and dysfunction during disease. Exp Eye Res. 2013 Dec; 117:1-3. doi: 10.1016/j.exer.2013.10.008. PMID: 24280033; PMCID: PMC4225770.

37. Dukovski BJ, Bračko A, Šare M, Pepic I, Lovric J. In vitro evaluation of stearylamine cationic nanoemulsions for improved ocular drug delivery. Acta Pharm. 2019 Dec 1;69(4):621-634. doi: 10.2478/acph-2019-0054. PMID: 31639085.

38. Hagbom M, Nordgren J, Nybom R, Hedlund KO, Wigzell H, Svensson L. Ionizing air affects influenza virus infectivity and prevents airborne-transmission. Sci Rep. 2015 Jun 23;5:11431. doi: 10.1038/srep11431. PMID: 26101102; PMCID: PMC4477231.

39. Peti W, Johnson MA, Herrmann T, Neuman BW, Buchmeier MJ, Nelson M, Joseph J, Page R, Stevens RC, Kuhn P, Wüthrich K. Structural genomics of the severe acute respiratory syndrome coronavirus: nuclear magnetic resonance structure of the protein nsP7. J Virol. 2005 Oct;79(20):12905-13. doi: 10.1128/JVI.79.20.12905-12913.2005. PMID: 16188992; PMCID: PMC1235862.

40. Tayyari F, Marchant D, Moraes TJ, Duan W, Mastrangelo P, Hegele RG. 2011. Identification of nucleolin as a cellular receptor for human respiratory syncytial virus. Nat. Med. 17:1132-1135.

41. Xu, X.-t. et al. Evolution of the novel coronavirus from the ongoing Wuhan outbreak and modeling of its spike protein for risk of human transmission. SCIENCE CHINA Life Sciences 63 (2020).

42. Zhao, Y., Zhao, Z., Wang, Y., Zhou, Y., Ma, Y., Zuo, W. Single-cell RNA expression profiling of ACE2, the putative receptor of Wuhan 2019-nCov. bioRxiv 2020.01.26.919985; doi: https://doi.org/10.1101/2020.01.26.919985.

43. Wu, K.-1., Li, W.-k., Peng, G.-q. & Li, F. Crystal structure of NL63 respiratory coronavirus receptor-binding domain complexed with its human receptor. Proc Natl Acad Sci U S A 106, 19970-19974 (2009).

44. Li, W. et al. The S proteins of human coronavirus NL63 and severe acute respiratory syndrome coronavirus bind overlapping regions of ACE2. Virology 367, 367-374 (2007).

45. He, L. et al. Expression of elevated levels of pro-inflammatory cytokines in SARS-CoV-infected ACE2+ cells in SARS patients: relation to the acute lung injury and pathogenesis of SARS. Journal of Pathology 210 (2006).

46. Zhou, P. et al. Discovery of a novel coronavirus associated with the recent pneumonia outbreak in humans and its potential bat origin. bioRxiv, 2020.2001.2022.914952, doi:10.1101/2020.01.22.914952 (2020).

47. Hamming I, Timens W, Bulthuis ML, Lely AT, Navis G, van Goor H. Tissue distribution of ACE2 protein, the functional receptor for SARS coronavirus. A first step in understanding SARS pathogenesis. J Pathol. 2004 Jun;203(2):631-7. doi: 10.1002/path.1570. PMID: 15141377.

48. Jia HP, Look DC, Shi L, Hickey M, Pewe L, Netland J, Farzan M, Wohlford-Lenane C, Perlman S, McCray PB Jr. ACE2 receptor expression and severe acute respiratory syndrome coronavirus infection depend on differentiation of human airway epithelia. J Virol. 2005 Dec;79(23):14614-21. doi: 10.1128/JVI.79.23.14614-14621.2005. PMID: 16282461; PMCID: PMC1287568.

49. Savaskan E, Loffler KU, Meier F, Muller-Spahn F, Flammer J, Meyer P. Immunohistochemical localization of angiotensin-converting enzyme, angiotensin II and AT1 receptor in human ocular tissues. Ophthalmic Res. 2004 Nov-Dec;36(6):312-20. doi: 10.1159/000081633. PMID: 15627831.

50. Chentoufi AA, Dasgupta G, Nesburn AB, Bettahi I, Binder NR, Choudhury ZS, Chamberlain WD, Wechsler SL, BenMohamed L. 2010. Nasolacrimal duct closure modulates ocular mucosal and systemic CD4( ) T-cell responses induced following topical ocular or intranasal immunization. Clin. Vaccine Immunol. 17:342-353.

51. Paulsen F. 2008. Functional anatomy and immunological interactions of ocular surface and adnexa.Dev. Ophthalmol. 41: 21-35.

52. Feucht HH, Zollner B, Schroter M, Altrogge H, Laufs R. 1995. Tear fluid of hepatitis C virus carriers could be infectious. J. Clin. Microbiol. 33: 2202-2203.

53. Fujikawa LS, Salahuddin SZ, Ablashi D, Palestine AG, Masur H, Nussenblatt RB, Gallo RC. 1986. HTLV-III in the tears of AIDS patients. Ophthalmology 93: 1479-1481.

54. Kalkan A, Ozden M, Yilmaz T, Demirdag K, Bulut Y, Ozdarendeli A. 2004. A case of mumps conjunctivitis: detection of the virus RNA by nested PCR in tear sample. Scand. J. Infect. Dis. 36: 697-700.

55. Kaufman HE, Azcuy AM, Varnell ED, Sloop GD, Thompson HW, Hill JM. 2005. HSV-1 DNA in tears and saliva of normal adults. Invest. Ophthalmol. Vis. Sci. 46: 241-247.

56. Kidd-Ljunggren K, Holmberg A, Blackberg J, Lindqvist B. 2006. High levels of hepatitis B virus DNA in body fluids from chronic carriers. J. Hosp. Infect. 64: 352-357.

57. https://www.cdc.gov/coronavirus/2019-ncov/hcp/guidance-prevent-spread.html?CDC_AA_refVal=https%3A%2F%2Fwww.cdc.gov%2Fcoronavirus%2F2019-ncov%2Fguidance-prevent-spread.html accessed 31 Jan. 2020.

58. https://www.cdc.gov/coronavirus/2019-ncov/hcp/infection-control.html?CDC_AA_refVal=https%3A%2F%2Fwww.cdc.gov%2Fcoronavirus%2F2019-ncov%2Finfection-control.html accessed 31January 2020.

59. Lu, Cheng-wei, Liu Xiu-fen, Jia Zhi-fang. 2019-nCoV transmission through the ocular surface must not be ignored The Lancet, https://www.thelancet.com/journals/lancet/article/PIIS0140-6736(20)30313-5/fulltext#articleInformation, accessed Feb. 8, 2020.

60. Dai X. Peking University Hospital Wang Guangfa disclosed treatment status on Weibo and suspected infection without wearing goggles. Jan. 22, 2020. http://www.bjnews.com.cn/news/2020/01/23/678189.html (accessed Feb. 7, 2020).

61. https://www.msn.com/en-us/news/world/chinese-doctor-who-issued-early-warning-on-virus-dies/ar-BBZJrw6?ocid=spartandhp, accessed Feb. 8, 2020.

62. https://www.aao.org/headline/alert-important-coronavirus-context accessed 16 Mar. 2020.

63. Li JO, Lam DSC, Chen Y, Ting DSW. Novel Coronavirus disease 2019 (COVID-19): The importance of recognising possible early ocular manifestation and using protective eyewear. Br J Ophthalmol. 2020 Mar;104(3):297-298. doi: 10.1136/bjophthalmol-2020-315994. PMID: 32086236.

64. She J, Jiang J, Ye L, Hu L, Bai C, Song Y. 2019 novel coronavirus of pneumonia in Wuhan, China: emerging attack and management strategies. Clin Transl Med. 2020 Feb 20;9(1):19. doi: 10.1186/s40169-020-00271-z. PMID: 32078069; PMCID: PMC7033263.

65. Mizumoto K, Chowell G. Estimating Risk for Death from 2019 Novel Coronavirus Disease, China, January-February 2020. Emerg Infect Dis. 2020 Mar 13;26(6). doi: 10.3201/eid2606.200233. Epub ahead of print. PMID: 32168464.

66. Baud D, Qi X, Nielsen-Saines K, Musso D, Pomar L, Favre G. Real estimates of mortality following COVID-19 infection. Lancet Infect Dis. 2020 Mar 12:S1473-3099(20)30195-X. doi: 10.1016/S1473-3099(20)30195-X. Epub ahead of print. PMID: 32171390.

67. Mahase, E. (2020). Coronavirus covid-19 has killed more people than SARS and MERS combined, despite lower case fatality rate. BMJ. https://doi. org/10.1136/bmj.m641.

68. Zumla A, Chan JF, Azhar EI, Hui DS, Yuen KY. Coronaviruses - drug discovery and therapeutic options. Nat Rev Drug Discov. 2016;15(5):327-347. doi:10.1038/nrd.2015.37.

69. Pillaiyar T, Meenakshisundaram S, Manickam M. Recent discovery and development of inhibitors targeting coronaviruses. Drug Discov Today. 2020 Jan 30:S1359-6446(20)30041-6. doi: 10.1016/j.drudis.2020.01.015. Epub ahead of print. PMID: 32006468.

70. de Wilde AH, Jochmans D, Posthuma CC, Zevenhoven-Dobbe JC, van Nieuwkoop S, Bestebroer TM, van den Hoogen BG, Neyts J, Snijder EJ. Screening of an FDA-approved compound library identifies four small-molecule inhibitors of Middle East respiratory syndrome coronavirus replication in cell culture. Antimicrob Agents Chemother. 2014 Aug;58(8):4875-84. doi: 10.1128/AAC.03011-14. Epub 2014 May 19. PMID: 24841269; PMCID: PMC4136071.

71. Martinez MA. Compounds with therapeutic potential against novel respiratory 2019 coronavirus. Antimicrob Agents Chemother. 2020 Mar 9:AAC.00399-20. doi: 10.1128/AAC.00399-20. Epub ahead of print. PMID: 32152082.

72. Kruse RL. Therapeutic strategies in an outbreak scenario to treat the novel coronavirus originating in Wuhan, China [version 2; peer review: 2 approved]. F1000Research 2020, 9:72 (https://doi.org/10.12688/f1000research.22211.2)

73. Wu C, Liu Y, Yang Y et al. Analysis of therapeutic targets for SARS-CoV-2 and discovery of potential drugs by computational methods. Acta Pharmaceutica Sinica B. Available online 27 Feb. 2020.

74. Gurwitz D. Angiotensin receptor blockers as tentative SARS-CoV-2 therapeutics. Drug Dev Res. 2020 Mar 4. doi: 10.1002/ddr.21656. Epub ahead of print. PMID: 32129518.

75. Li W, Moore MJ, Vasilieva N, Sui J, Wong SK, Berne MA, Somasundaran M, Sullivan JL, Luzuriaga K, Greenough TC, Choe H, Farzan M. Angiotensin-converting enzyme 2 is a functional receptor for the SARS coronavirus. Nature. 2003 Nov 27;426(6965):450-4. doi: 10.1038/nature02145. PMID: 14647384.

76. Abraham HM, White CM, White WB. The comparative efficacy and safety of the angiotensin receptor blockers in the management of hypertension and other cardiovascular diseases. Drug Saf. 2015 Jan;38(1):33-54. doi: 10.1007/s40264-014-0239-7. PMID: 25416320; PMCID: PMC4303500.

77. Natesh R, Schwager SL, Sturrock ED, Acharya KR. Crystal structure of the human angiotensin-converting enzyme-lisinopril complex. Nature. 2003 Jan 30;421(6922):551-4. doi: 10.1038/nature01370. Epub 2003 Jan 19. PMID: 12540854.

78. Wu K, Chen L, Peng G, Zhou W, Pennell CA, Mansky LM, Geraghty RJ, Li F. A virus-binding hot spot on human angiotensin-converting enzyme 2 is critical for binding of two different coronaviruses. J Virol. 2011 Jun;85(11):5331-7. doi: 10.1128/JVI.02274-10. Epub 2011 Mar 16. PMID: 21411533; PMCID: PMC3094985.

79. Towler P, Staker B, Prasad SG, Menon S, Tang J, Parsons T, Ryan D, Fisher M, Williams D, Dales NA, Patane MA, Pantoliano MW. ACE2 X-ray structures reveal a large hinge-bending motion important for inhibitor binding and catalysis. J Biol Chem. 2004 Apr 23;279(17): 17996-8007. doi: 10.1074/jbc.M311191200. Epub 2004 Jan 30. PMID: 14754895.

80. Savarino A, Boelaert JR, Cassone A, Majori G, Cauda R. Effects of chloroquine on viral infections: an old drug against today’s diseases? Lancet Infect Dis. 2003 Nov;3(11):722-7. doi: 10.1016/s1473-3099(03)00806-5. PMID: 14592603.

81. Vincent MJ, Bergeron E, Benjannet S, Erickson BR, Rollin PE, Ksiazek TG, Seidah NG, Nichol ST. Chloroquine is a potent inhibitor of SARS coronavirus infection and spread. Virol J. 2005 Aug 22;2:69. doi: 10.1186/1743-422X-2-69. PMID: 16115318; PMCID: PMC1232869.

82. Keyaerts E, Vijgen L, Maes P, Neyts J, Van Ranst M. In vitro inhibition of severe acute respiratory syndrome coronavirus by chloroquine. Biochem Biophys Res Commun. 2004 Oct 8;323(1):264-8. doi: 10.1016/j.bbrc.2004.08.085. PMID: 15351731.

83. Savarino A, Di Trani L, Donatelli I, Cauda R, Cassone A. New insights into the antiviral effects of chloroquine. Lancet Infect Dis. 2006 Feb;6(2):67-9. doi: 10.1016/S1473-3099(06)70361-9. PMID: 16439323.

84. Yao X, Ye F, Zhang M, Cui C, Huang B, Niu P, Liu X, Zhao L, Dong E, Song C, Zhan S, Lu R, Li H, Tan W, Liu D. In Vitro Antiviral Activity and Projection of Optimized Dosing Design of Hydroxychloroquine for the Treatment of Severe Acute Respiratory Syndrome Coronavirus 2 (SARS-CoV-2). Clin Infect Dis. 2020 Mar 9:ciaa237. doi: 10.1093/cid/ciaa237. Epub ahead of print. PMID: 32150618.

85. Liu J, Cao R, Xu M et al. Hydroxychloroquine, a less toxic derivative of chloroquine, is effective in inhibiting SARS-CoV-2 infection in vitro. Cell Discovery 2020;6:16-19.

86. https://www.medscape.com/viewarticle/927033 accessed 19 Mar. 2020.

87. Li X, He Z, Chen L, Li Y, Li Q, Zhao S, Tao Z, Hu W, Qin L, Chen X. Synergy of the antiretroviral protease inhibitor indinavir and chloroquine against malaria parasites in vitro and in vivo. Parasitol Res. 2011 Dec;109(6):1519-24. doi: 10.1007/s00436-011-2427-z. Epub 2011 May 3. PMID: 21537980.

88. Yusuf IH, Sharma S, Luqmani R, Downes SM. Hydroxychloroquine retinopathy. Eye (Lond). 2017 Jun;31(6):828-845. doi: 10.1038/eye.2016.298. Epub 2017 Mar 10. PMID: 28282061; PMCID: PMC5518824.

89. Sauer L, Calvo CM, Vitale AS, Henrie N, Milliken CM, Bernstein PS. Imaging of Hydroxychloroquine Toxicity with Fluorescence Lifetime Imaging Ophthalmoscopy. Ophthalmol Retina. 2019 Oct;3(10):814-825. doi: 10.1016/j.oret.2019.04.025. Epub 2019 May 2. PMID: 31345727.

90. Melles RB, Marmor MF. The risk of toxic retinopathy in patients on long-term hydroxychloroquine therapy. JAMA Ophthalmol 2014;132:1453-60.

91. Pasaoglu I, Onmez FE. Macular toxicity after short-term hydroxychloroquine therapy. Indian J Ophthalmol. 2019 Feb;67(2):289-292. doi: 10.4103/ijo.IJO_732_18. PMID: 30672499; PMCID: PMC6376838.

92. Chiang E, Jampol LM, Fawzi AA. Retinal toxicity found in a patient with systemic lupus erythematosus prior to 5 years of treatment with hydroxychloroquine. Rheumatology (Oxford) 2014;53:2001.

93. Shroyer NF, Lewis RA, Lupski JR. Analysis of the ABCR (ABCA4) gene in 4-aminoquinoline retinopathy: Is retinal toxicity by chloroquine and hydroxychloroquine related to Stargardt disease? Am J Ophthalmol 2001;131:761-6.

94. Hedner T, Samulesson O, Währborg P, Wadenvik H, Ung KA, Ekbom A. Nabumetone: Therapeutic use and safety profile in the management of osteoarthritis and rheumatoid arthritis. Drugs 2004;64:2315-43; discussion 2344-5.

95. Hernandez Bel L, Monferrer Adsuara C, Hernandez Garfella M, Cervera Taulet E. Early macular toxicity following 2 months of hydroxychloroquine therapy. Arc Soc Esp Oftalmol 2018;93:20-1.

96. Marmor MF, Kellner U, Lai TY, Melles RB, Mieler WF. Recommendations on Screening for Chloroquine and Hydroxychloroquine Retinopathy (2016 Revision). Ophthalmology 2016; 123: 1386-1394.

97. Giocanti-Auregan A, Couturier A, Girmens JF, Le Mer Y, Massamba N, Barreau E, Audo I; DHU Vision Handicaps task force retine. Differences ethniques parmi les patients souffrant de toxicité maculaire aux antipaludeens de synthèse [Variability of chloroquine and hydroxychloroquine retinopathy among various ethnicities]. J Fr Ophtalmol. 2018 Apr;41(4):363-367. French. doi: 10.1016/j.jfo.2017.08.014. Epub 2018 Apr 17. PMID: 29678344.

98. Xue J, Moyer A, Peng B, Wu J, Hannafon BN, Ding WQ. Chloroquine is a zinc ionophore. PLoS One. 2014 Oct 1;9(10):e109180. doi: 10.1371/journal.pone.0109180. PMID: 25271834; PMCID: PMC4182877.

99. te Velthuis AJ, van den Worm SH, Sims AC, Baric RS, Snijder EJ, van Hemert MJ. Zn(2+) inhibits coronavirus and arterivirus RNA polymerase activity in vitro and zinc ionophores block the replication of these viruses in cell culture. PLoS Pathog. 2010 Nov 4;6(11):e1001176. doi: 10.1371/journal.ppat.1001176. PMID: 21079686; PMCID: PMC2973827.

100. Kaushik N, Subramani C, Anang S, et al. Zinc Salts Block Hepatitis E Virus Replication by Inhibiting the Activity of Viral RNA-Dependent RNA Polymerase. J Virol. 2017;91(21):e00754-17.

101. https://www.youtube.com/watch?v=U7F1cnWup9M accessed 21 Mar. 2020

102. Cortegiania A, Ingoglia G, Ippolitoa A et al.A systematic review on the efficacy and safety of chloroquine for the treatment of COVID-19. Journal of Critical Care. 2020 (in press).

103. Toimela T, Tahti H, Salminen L. Retinal pigment epithelium cell culture as a model for evaluation of the toxicity of tamoxifen and chloroquine. Ophthalmic Res 1995;27(Suppl 1): 150-153.

104. Seo BR, Lee SJ, Cho KS, Yoon YH, Koh JY. The zinc ionophore clioquinol reverses autophagy arrest in chloroquine-treated ARPE-19 cells and in APP/mutant presenilin-1-transfected Chinese hamster ovary cells. Neurobiol Aging. 2015 Dec;36(12):3228-3238. doi: 10.1016/j.neurobiolaging.2015.09.006. Epub 2015 Sep 16. PMID: 26453000.

105. Rajapakse D, Curtis T, Chen M, Xu H. Zinc Protects Oxidative Stress-Induced RPE Death by Reducing Mitochondrial Damage and Preventing Lysosome Rupture. Oxid Med Cell Longev. 2017;2017:6926485. doi: 10.1155/2017/6926485. Epub 2017 Nov 14. PMID: 29348791; PMCID: PMC5733978.

106. Bhavsar, Ankita Samir, Samir G. Bhavsar and Sunita M. Jain. Evaluation of the effects of chloroquine phosphate eye drops in patients with dry eye syndrome. International Journal of Biomedical and Advance Research 2011;2:198-214. doi.org/10.7439 /ijbar.v2i6.34.

107. Titiyal JS, Kaur M, Falera R, Bharghava A, Sah R, Sen S. Efficacy and Safety of Topical Chloroquine in Mild to Moderate Dry Eye Disease. Curr Eye Res. 2019 Dec;44(12):1306-1312. doi: 10.1080/02713683.2019.1641824. Epub 2019 Jul 15. PMID: 31283891.

108. Watkins RW, Baum T, Cedeno K, Smith EM, Yuen PH, Ahn HS, Barnett A. Topical ocular hypotensive effects of the novel angiotensin converting enzyme inhibitor SCH 33861 in conscious rabbits. J Ocul Pharmacol. 1987 Winter;3(4):295-307. doi: 10.1089/jop.1987.3.295. PMID: 3503919.

109. Shah GB, Sharma S, Mehta AA, Goyal RK. Oculohypotensive effect of angiotensin-converting enzyme inhibitors in acute and chronic models of glaucoma. J Cardiovasc Pharmacol. 2000 Aug;36(2):169-75. doi: 10.1097/00005344-200008000-00005. PMID: 10942157.

110. Watkins RW, Baum T, Tedesco RP, Pula K, Barnett A. Systemic effects resulting from topical ocular administration of SCH 33861, a novel ACE inhibitor ocular hypotensive agent. J Ocul Pharmacol. 1988 Summer;4(2):93-100. doi: 10.1089/jop.1988.4.93. PMID: 3049862.

111. Burnier M, Maillard M. The comparative pharmacology of angiotensin II receptor antagonists. Blood Press Suppl. 2001;1:6-11. PMID: 11333013.

112. https://www.drugs.com/uk/zinc-sulphate-eye-drops-bp-0-25-leaflet.html accessed 21 Mar. 2020.

113. Hubbard GB, Herron BE, Andrews JS, Elliott JH. Influence of topical and oral zinc upon corneal wound healing. Br J Ophthalmol. 1969 Jun;53(6):407-11. doi: 10.1136/bjo.53.6.407. PMID: 5794958; PMCID: PMC1214584.

114. ARRIGONI L, FISCHER L, TOZER GA. OPHTHALMIC ZINC SULFATE SOLUTIONS: BUFFERED, ISOTONIC AND PRESERVED. Arch Ophthalmol. 1941;26(5):852-858. doi:10.1001/archopht.1941.00870170144011.

115. https://patents.google.com/patent/US20020123482 accessed 21 Mar. 2020

116. Ishiyama Y, Gallagher PE, Averill DB, Tallant EA, Brosnihan KB, Ferrario CM. Upregulation of angiotensin-converting enzyme 2 after myocardial infarction by blockade of angiotensin II receptors. Hypertension. 2004 May;43(5):970-6. doi: 10.1161/01.HYP.0000124667.34652.1a. Epub 2004 Mar 8. PMID: 15007027.

117. Leung C. Clinical features of deaths in the novel coronavirus epidemic in China. Rev Med Virol. 2020 Mar 16:e2103. doi: 10.1002/rmv.2103. Epub ahead of print. PMID: 32175637.

118. Gautret C, Lagier J-C, Parola P et al. Hydroxychloroquine and azithromycin as a treatment of COVID-19: results of an open-label non-randomized clinical trial. International Journal of Antimicrobial Agents - In Press 17 Mar. 2020 - DOI: 10.1016/j.ijantimicag.2020.105949.

119. Opitz DL, Harthan JS. Review of Azithromycin Ophthalmic 1% Solution (AzaSite(®)) for the Treatment of Ocular Infections. Ophthalmol Eye Dis. 2012 Feb 23;4:1-14. doi: 10.4137/OED.S7791. PMID: 23650453; PMCID: PMC3619494.

120. Amza A, Goldschmidt P, Einterz E, Huguet P, Olmiere C, Bensaid P, Bella-Assumpta L. Elimination of active trachoma after two topical mass treatments with azithromycin 1.5% eye drops. PLoS Negl Trop Dis. 2010 Nov 23;4(11):e895. doi: 10.1371/journal.pntd.0000895. PMID: 21124889; PMCID: PMC2990706.

121. Khalid M, Khan B, Al Rabiah F, Alismaili R, Saleemi S, Rehan-Khaliq AM, Weheba I, Al Abdely H, Halim M, Nadri QJ, Al Dalaan AM, Zeitouni M, Butt T, Al Mutairy E. Middle Eastern Respiratory Syndrome Corona Virus (MERS CoV): case reports from a tertiary care hospital in Saudi Arabia. Ann Saudi Med. 2014 Sep-Oct;34(5):396-400. doi: 10.5144/0256-4947.2014.396. PMID: 25827696; PMCID: PMC6074560.

122. Krilis M, Tsang H, Coroneo M. Treatment of conjunctival and corneal epithelial neoplasia with retinoic acid and topical interferon alfa-2b: long-term follow-up. Ophthalmology. 2012 Oct; 119(10): 1969-73. doi: 10.1016/j.ophtha.2012.03.045. Epub 2012 Jun 14. PMID: 22704834.

123. Yuan S. Statins May Decrease the Fatality Rate of Middle East Respiratory Syndrome Infection. mBio. 2015 Aug 11;6(4):e01120. doi: 10.1128/mBio.01120-15. PMID: 26265720; PMCID: PMC4542194.

124. Ooi KG, Wakefield D, Billson FA, Watson SL. Efficacy and Safety of Topical Atorvastatin for the Treatment of Dry Eye Associated with Blepharitis: A Pilot Study. Ophthalmic Res. 2015;54(1):26-33. doi: 10.1159/000367851. Epub 2015 Jun 5. PMID: 26068735.

125. Ulian M-E, Linas S-L. Role of receptor cycling in the regulation of angiotensin II surface receptor number and angiotensin II uptake in rat vascular smooth muscle cells. J Clin Invest. (1989) 84:840-846.

126. Kalenga MK, De Gasparo M, Thomas K, De Hertogh R. Down-regulation of angiotensin AT1 receptor by progesterone in human placenta. J Clin Endocrinol Metab. 1996 Mar;81(3):998-1002. doi: 10.1210/jcem.81.3.8772564. PMID: 8772564.

127. Al-Bari MAA. Targeting endosomal acidification by chloroquine analogs as a promising strategy for the treatment of emerging viral diseases. Pharmacol Res Perspect. 2017 Jan 23;5(1):e00293. doi: 10.1002/prp2.293. PMID: 28596841; PMCID: PMC5461643.

128. Shrinidh Joshi, Narayanaganesh Balasubramanian, Goutham Vasam, and Yagna PR Jarajapu. Angiotensin converting enzyme versus angiotensin converting enzyme-2 selectivity of MLN-4760 and DX600 in human and murine bone marrow-derived cells, Eur J Pharmacol. 2016 March 5; 774: 25-33. doi:10.1016/j.ejphar.2016.01.007.

129. Chu CM, Poon LL, Cheng VC, Chan KS, Hung IF, Wong MM, Chan KH, Leung WS, Tang BS, Chan VL, Ng WL, Sim TC, Ng PW, Law KI, Tse DM, Peiris JS, Yuen KY. Initial viral load and the outcomes of SARS. CMAJ. 2004 Nov 23;171(11):1349-52. doi: 10.1503/cmaj.1040398. PMID: 15557587; PMCID: PMC527336.

130. Mark B. Abelson, Lisa M. Smith, Vasoconstrictors: Myths and Realities, https://www.reviewofophthalmology.com/article/vasoconstrictors-myths-and-realities, Review of Ophthalmology, Aug. 9, 2012.

131. Eccles, R., Winther, B., Johnston, S. et al. Efficacy and safety of iotacarrageenan nasal spray versus placebo in early treatment of the common cold in adults: the ICICC trial. Respir Res 16, 121 (2015). https://doi.org/10.1186/sl2931-015-0281-8.

132. Christine Graf, et al., Development of a nasal spray containing xylometazoline hydrochloride and iotacarrageenan for the symptomatic relief of nasal congestion caused by rhinitis and sinusitis, International Journal of General Medicine 2018:11 275-283.

### INCORPORATION BY REFERENCE

Various references such as patents, patent applications, and publications are cited herein, the disclosures of which are hereby incorporated by reference herein in their entireties.

We claim:

1. A composition, comprising:
i) an effective amount of hydroxychloroquine or chloroquine, or a pharmaceutically acceptable salt thereof;
ii) an effective amount of a source of zinc ion; and
iii) a pharmaceutically acceptable excipient, carrier or diluent;
wherein the composition is an ophthalmic, an inhalation, or a topical composition.

2. A composition, comprising:
i) an effective amount of hydroxychloroquine or chloroquine, or a pharmaceutically acceptable salt thereof;
ii) an effective amount of azithromycin; and
iii) a pharmaceutically acceptable excipient, carrier or diluent;
wherein the composition is an ophthalmic, an inhalation, or a topical composition.

3. A composition, comprising:
i) an effective amount of hydroxychloroquine or chloroquine, or a pharmaceutically acceptable salt thereof;
ii) an effective amount of a source of zinc ion;
iii) an effective amount of azithromycin; and
iv) a pharmaceutically acceptable excipient, carrier or diluent;
wherein the composition is an ophthalmic, an inhalation, or a topical composition.

4. A composition, comprising:
i) an effective amount of iota-carrageenan or a pharmaceutically acceptable salt thereof; and
ii) a pharmaceutically acceptable excipient, carrier or diluent;
wherein the composition is an ophthalmic, an inhalation, or a topical composition.

5. A composition, comprising:
i) an effective amount of iota-carrageenan or a pharmaceutically acceptable salt thereof;
ii) an effective amount of hydroxychloroquine or chloroquine, or a pharmaceutically acceptable salt thereof; and
iii) a pharmaceutically acceptable excipient, carrier or diluent;
wherein the composition is an ophthalmic, an inhalation, or a topical composition.

6. A composition, comprising:
i) an effective amount of iota-carrageenan or a pharmaceutically acceptable salt thereof;
ii) an effective amount of hydroxychloroquine or chloroquine, or a pharmaceutically acceptable salt thereof;
iii) an effective amount of azithromycin; and
iv) a pharmaceutically acceptable excipient, carrier or diluent;
wherein the composition is an ophthalmic, an inhalation, or a topical composition.

7. A composition, comprising:
i) an effective amount of iota-carrageenan or a pharmaceutically acceptable salt thereof;
ii) an effective amount of hydroxychloroquine or chloroquine, or a pharmaceutically acceptable salt thereof;
iii) an effective amount of azithromycin;
iv) an effective amount of a source of zinc ion; and
v) a pharmaceutically acceptable excipient, carrier or diluent;
wherein the composition is an ophthalmic, an inhalation, or a topical composition.

8. The composition of claim 1, wherein the composition further comprises an effective amount of iota-carrageenan or a pharmaceutically acceptable salt thereof.

9. The composition of any one of claims 1-8, wherein the composition further comprises an effective amount of an ACE2 inhibitor.

10. The composition of any one of claims 1-9, wherein the composition further comprises an effective amount of a vasoconstrictor.

11. The composition of any one of claims 1-3 or 5-10, wherein the effective amount of the hydroxychloroquine or the chloroquine, or pharmaceutically acceptable salt thereof, is an amount suitable for local administration to an eye, a nose, a face, and/or an oral cavity of a subj ect.

12. The composition of any one of claims 1-3 or 5-11, wherein the effective amount of the hydroxychloroquine or the chloroquine, or pharmaceutically acceptable salt thereof, is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said hydroxychloroquine or said chloroquine, respectively.

13. The composition of any one of claim 1, claim 3, or claims 7-12, wherein the effective amount of the source of zinc ion is an amount suitable for local administration to an eye, a nose, a face, and/or an oral cavity of a subject.

14. The composition of any one of claim 1, claim 3, or claims 7-13, wherein the effective amount of the source of zinc ion is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said source of zinc ion.

15. The composition of any one of claim 1, claim 3, or claims 7-14, wherein the effective amount of the source of zinc ion is a prophylactically effective amount of said source of zinc ion.

16. The composition of any one of claim 1, claim 3, or claims 7-14, wherein the effective amount of the source of zinc ion is a therapeutically effective amount of said source of zinc ion.

17. The composition of any one of claim 1, claim 3, or claims 7–16, wherein the source of zinc ion is a pharmaceutically acceptable salt.

18. The composition of any one of claim 1, claim 3, or claims 7-17, wherein the source of zinc ion is an ophthalmically acceptable salt.

19. The composition of any one of claim 1, claim 3, or claims 7-18, wherein the source of zinc ion is present in said composition at a concentration in the range of between about 0.05-0.50 wt%.

20. The composition of any one of claim 1, claim 3, or claims 7-19, wherein the source of zinc ion is present in said composition at a concentration of about 0.05 wt.%, about 0.10 wt.%, about 0.15 wt.%, about 0.20 wt.%, about 0.25 wt.%, about 0.30 wt.%, about 0.35 wt.%, about 0.40 wt.%, about 0.45 wt.%, or about 0.50 wt.%.

21. The composition of any one of claim 1, claim 3, or claims 7-20, wherein the source of zinc ion is present in said composition at a concentration in the range of between about 1-30 uM.

22. The composition of any one of claim 1, claim 3, or claims 7-21, wherein the source of zinc ion is present in said composition at a concentration of about 1 uM, about 2 uM, about 5 uM, about 10 uM, about 15 uM, about 20 uM, about 25 uM, or about 30 uM.

23. The composition of any one of claim 1, claim 3, or claims 7-22, wherein the source of zinc ion is zinc sulfate or zinc chloride.

24. The composition of any one of claim 1, claim 3, or claims 7-23, wherein the source of zinc ion is zinc sulfate.

25. The composition of any one of claim 1, claim 3, or claims 7-24, wherein the source of zinc ion is zinc chloride.

26. The composition of any one of claims 2-3 or claims claims 6-25, wherein the effective amount of the azithromycin is an amount suitable for local administration to an eye, a nose, a face, and/or an oral cavity of a subject.

27. The composition of any one of claims 2-3 or claims claims 6-26, wherein the effective amount of the azithromycin is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said azithromycin.

28. The composition of any one of claims 2-3 or claims claims 6-27, wherein the effective amount of the azithromycin is a prophylactically effective amount of said azithromycin.

29. The composition of any one of claims 2-3 or claims claims 6-28, wherein the effective amount of the azithromycin is a therapeutically effective amount of said azithromycin.

30. The composition of any one of claims 2-3 or claims claims 6-29, wherein the azithromycin is present in said composition at a concentration in the range of between about 0.5-3 wt%.

31. The composition of any one of claims 2-3 or claims claims 6-30, wherein the azithromycin is present in said composition at a concentration of about 0.5 wt.%, about 1.0 wt.%, about 1.5 wt.%, about 2.0 wt.%, about 2.5 wt.%, or about 3.0 wt.%.

32. The composition of any one of claims 2-3 or claims claims 6-31, wherein the azithromycin is present in said composition at a concentration in the range of between about 1-30 uM.

33. The composition of any one of claims 2-3 or claims claims 6-32, wherein the azithromycin is present in said composition at a concentration of about 1 uM, about 2 uM, about 5 uM, about 10 uM, about 15 uM, about 20 uM, about 25 uM, or about 30 uM.

34. The composition of any one of claims 4-3, wherein the effective amount of the iota-carrageenan or pharmaceutically acceptable salt thereof is an amount suitable for local administration to an eye, a nose, a face, and/or an oral cavity of a subject.

35. The composition of any one of claims 4-34, wherein the effective amount of the iota-carrageenan or pharmaceutically acceptable salt thereof is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said iota-carrageenan or pharmaceutically acceptable salt thereof.

36. The composition of any one of claims 4-35, wherein the effective amount of the iota-carrageenan or pharmaceutically acceptable salt thereof is a prophylactically effective amount of said iota-carrageenan or pharmaceutically acceptable salt thereof.

37. The composition of any one of claims 4-35, wherein the effective amount of the iota-carrageenan or pharmaceutically acceptable salt thereof is a therapeutically effective amount of said iota-carrageenan or pharmaceutically acceptable salt thereof.

38. The composition of any one of claims 4-37, wherein the iota-carrageenan or pharmaceutically acceptable salt thereof is present in said composition at a concentration in the range of between about 1.0-1.5 mg/mL.

39. The composition of any one of claims 4-38, wherein the iota-carrageenan or pharmaceutically acceptable salt thereof is present in said composition at a concentration of about 1.2 mg/mL.

40. The composition of any one of claims 9-39, wherein the effective amount of the ACE2 inhibitor is an amount suitable for local administration to an eye, a nose, a face, and/or an oral cavity of a subject.

41. The composition of any one of claims 9-40, wherein the effective amount of the ACE2 inhibitor is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said ACE2 inhibitor.

42. The composition of any one of claims 9-41, wherein the effective amount of the ACE2 inhibitor is a prophylactically effective amount of said ACE2 inhibitor.

43. The composition of any one of claims 9-41, wherein the effective amount of the ACE2 inhibitor is a therapeutically effective amount of said ACE2 inhibitor.

44. The composition of any one of claims 9-43, wherein the ACE2 inhibitor is present in said composition at a concentration in the range of between about 0.01-3 wt%.

45. The composition of any one of claims 9-44, wherein the ACE2 inhibitor is present in said composition at a concentration of about 0.1 wt.%, 0.5 wt.%, about 1.0 wt.%, about 1.5 wt.%, about 2.0 wt.%, about 2.5 wt.%, or about 3.0 wt.%.

46. The composition of any one of claims 9-45, wherein the ACE2 inhibitor is MLN-4760 or DX600.

47. The composition of any one of claims 10-46, wherein the effective amount of the vasoconstrictor is an amount suitable for local administration to an eye, a nose, a face, and/or an oral cavity of a subject.

48. The composition of any one of claims 10-47, wherein the effective amount of the vasoconstrictor is 2 fold, 3 fold, 4 fold, 5 fold, 6 fold, 7 fold, 8 fold, 9 fold, 10 fold, 20 fold, 50 fold, 100 fold, 500 fold, 1,000 fold, 10,000 fold, or 100,000 fold, less than a systemically effective amount of said vasoconstrictor.

49. The composition of any one of claims 10-48, wherein the effective amount of the vasoconstrictor is a prophylactically effective amount of said vasoconstrictor.

50. The composition of any one of claims 10-48, wherein the effective amount of the vasoconstrictor is a therapeutically effective amount of said vasoconstrictor.

51. The composition of any one of claims 10-50, wherein the vasoconstrictor is vasoconstrictor is a sympathomimetic amine or a sympathomimetic imidazole.

52. The composition of any one of claims 10-51, wherein the vasoconstrictor is vasoconstrictor is xylometazoline, naphazoline, tetrahydrozoline, phenylephrine and oxymetazoline, or brimonidine, or a pharmaceutically acceptable salts thereof.

53. The composition of any one of claims 10-52, wherein the vasoconstrictor is xylometazoline hydrochloride.

54. The composition of any one of claims 10-53, wherein the xylometazoline hydrochloride is present in said composition at a concentration in the range of between about 0.25-1.0 mg/mL.

55. The composition of any one of claims 10-54, wherein the xylometazoline hydrochloride is present in said composition at a concentration of about 0.5 mg/mL.

56. The composition of any one of claims 1-3 or claims 5-55, wherein the composition comprises the hydroxychloroquine or pharmaceutically acceptable salt thereof.

57. The composition of claim 56, wherein the composition comprises the effective amount of the hydroxychloroquine or pharmaceutically acceptable salt thereof is a prophylactically effective amount of said hydroxychloroquine.

58. The composition of claim 56, wherein the composition comprises the effective amount of the hydroxychloroquine or pharmaceutically acceptable salt thereof is a therapeutically effective amount of said hydroxychloroquine.

59. The composition of any one of claims 1-3 or claims 5-58, wherein the hydroxychloroquine or pharmaceutically acceptable salt thereof is present in said composition at a concentration in the range of between about 0.001-0.10 wt%.

60. The composition of any one of claims 1-3 or claims 5-59, wherein the hydroxychloroquine or pharmaceutically acceptable salt thereof is present in said composition at a concentration of about 0.001 wt.%, about 0.005 wt.%, about 0.01 wt.%, about 0.02 wt.%, about 0.03 wt.%, about 0.04 wt.%, about 0.05 wt.%, about 0.06 wt.%, about 0.07 wt.%, about 0.08 wt.%, about 0.09 wt.%, or about 0.10 wt.%.

61. The composition of any one of claims 1-3 or claims 5-60, wherein the hydroxychloroquine or pharmaceutically acceptable salt thereof is present in said composition at a concentration in the range of between about 1-30 uM.

62. The composition of any one of claims 1-3 or claims 5-61, wherein the hydroxychloroquine or pharmaceutically acceptable salt thereof is present in said composition at a concentration of about 1 uM, about 2 uM, about 5 uM, about 10 uM, about 15 uM, about 20 uM, about 25 uM, or about 30 uM.

63. The composition of any one of claims 1-3 or claims 5-62, wherein the hydroxychloroquine or pharmaceutically acceptable salt thereof is hydroxychloroquine phosphate or hydroxychloroquine sulfate.

64. The composition of any one of claims 1-3 or claims 5-55, wherein the composition comprises the chloroquine or pharmaceutically acceptable salt thereof.

65. The composition of claim 64, wherein the composition comprises the effective amount of the chloroquine or pharmaceutically acceptable salt thereof is a prophylactically effective amount of said chloroquine.

66. The composition of claim 64, wherein the composition comprises the effective amount of the chloroquine or pharmaceutically acceptable salt thereof is a therapeutically effective amount of said chloroquine.

67. The composition of any one of claims 1-3, claims 5-55, or claims 64-66, wherein the chloroquine or pharmaceutically acceptable salt thereof is present in said composition at a concentration in the range of between about 0.001-0.10 wt%.

68. The composition of any one of claims 1-3, claims 5-55, or claims 64-67, wherein the chloroquine or pharmaceutically acceptable salt thereof is present in said composition at a concentration of about 0.001 wt.%, about 0.005 wt.%, about 0.01 wt.%, about 0.02 wt.%, about 0.03 wt.%, about 0.04 wt.%, about 0.05 wt.%, about 0.06 wt.%, about 0.07 wt.%, about 0.08 wt.%, about 0.09 wt.%, or about 0.10 wt.%.

69. The composition of any one of claims 1-3, claims 5-55, or claims 64-68, wherein the chloroquine or pharmaceutically acceptable salt thereof is present in said composition at a concentration in the range of between about 1-30 uM.

70. The composition of any one of claims 1-3, claims 5-55, or claims 64-69, wherein the chloroquine or pharmaceutically acceptable salt thereof is present in said composition at a concentration of about 1 uM, about 2 uM, about 5 uM, about 10 uM, about 15 uM, about 20 uM, about 25 uM, or about 30 uM.

71. The composition of any one of claims 1-3, claims 5-55, or claims 64-70, wherein the chloroquine or pharmaceutically acceptable salt thereof is chloroquine phosphate.

72. The composition of any one of claims 1-71, wherein the composition further comprises chlorpromazine, loperamide, indinavir, and/or lopinavir.

73. The composition of any one of claims 1-72, wherein the composition is a topical composition.

74. The composition of any one of claims 1-73, wherein the composition is an ophthalmic composition.

75. The composition of any one of claims 1-74, wherein the composition is an inhalation composition.

76. The composition of any one of claims 1-75, wherein the composition is suitable for oral administration.

77. The composition of any one of claims 1-76, wherein the composition is a solution, a spray, an aerosol, a gel, or a lotion.

78. The composition of any one of claims 1-77, wherein the composition is an aqueous composition.

79. The composition of any one of claims 1-78, wherein the composition is a sterile solution.

80. The composition of any one of claims 1-79, wherein the composition is a liquid spray.

81. The composition of any one of claims 1-80, wherein the composition is an eye drop composition.

82. A method of treating a subject exposed to a virus, comprising administering to a subject exposed to a virus an effective amount of the composition of any one of claims 1-81, wherein the virus is a virus that enters or infects the subject via association with or binding to an ACE2 receptor in the subject, or the virus is a coronavirus, for example, the virus is a COVID-19 virus.

83. A method of preventing or treating a viral infection in a subject, comprising administering to a subject exposed to a virus an effective amount of the composition of any one of claims 1-81, wherein the virus is a virus that enters or infects the subject via association with or binding to an ACE2 receptor in the subject, or the virus is a coronavirus, for example, the virus is a COVID-19 virus.

84. A method of prophylactically treating, comprising administering to a subject prior to exposure to a virus an effective amount of the composition of any one of claims 1-81, wherein the virus is a virus that enters or infects the subject via association with or binding to an ACE2 receptor in the subject, or the virus is a coronavirus, for example, the virus is a COVID-19 virus.

85. The method of claim 84, wherein the subject prior to exposure to a virus does not have symptoms associated with a viral infection, wherein the virus is a virus that enters or infects the subject via association with or binding to an ACE2 receptor in the subject, or the virus is a coronavirus, for example, the virus is a COVID-19 virus.

86. The method of any one of claims 82-85, wherein the composition is administered topically.

87. The method of any one of claims 82-86, wherein the composition is administered ophthalmically.

88. The method of any one of claims 82-87, wherein the composition is administered via inhalation.

89. The method of any one of claims 82-88, wherein the composition is administered to an ocular surface and/or eyelid of the subject.

90. The method of any one of claims 82-89, wherein the composition is administered to the subject’s face.

91. The method of any one of claims 82-90, wherein the composition is administered in a nostril of the subject.

92. The method of any one of claims 82-91, wherein the administered composition is a topical composition.

93. The method of claim 82, wherein the administered topical composition is suitable for oral administration.

94. The method of any one of claims 82-93, wherein the administered composition is an ophthalmic composition.

95. The method of claim 94, wherein the administered ophthalmic composition is suitable for oral administration.

96. The method of any one of claims 82-95, wherein the administered composition is an inhalation composition.

97. The method of claim 96, wherein the inhalation composition is administered via a puffer device suitable for inhalation.

98. The method of claim 96 or claim 97, wherein the inhalation composition is administered as a spray or as an aerosol by inhalation via the puffer device.

99. The method of any one of claims 96-98, wherein the inhalation composition is further administered topically to an ocular surface, an eyelid, in a nostril, or to the face of the subject via spraying the contents within said puffer device.

100. The method of any one of claims 82-99, wherein the administered inhalation composition is suitable for oral administration.

101. The method of any one of claims 82-100, wherein the administered composition is further administered to the surface of the subject’s oral cavity.

102. The method of any one of claims 82-101, wherein the exposed subject does not have symptoms associated with a viral infection, wherein the viral infection is a viral infection resulting from entering or infecting a subject via association with or binding to an ACE2 receptor in the subject, or the viral infection is a coronavirus infection, for example, the viral infection is a COVID-19 infection.

103. The method of any one of claims 82-102, wherein the method is a preventative treatment of the subject exposed to the virus to avoid contracting a viral infection, wherein the virus is a virus that enters or infects the subject via association with or binding to an ACE2 receptor in the subject, or the virus is a coronavirus, for example, the virus is a COVID-19 virus.

104. The method of any one of claims 82-103, wherein the method is a prophylactic treatment of the subject exposed to the virus, wherein the virus is a virus that enters or infects the subject via association with or binding to an ACE2 receptor in the subject, or the virus is a coronavirus, for example, the virus is a COVID-19 virus.

105. The method of any one of claims 82-101, wherein the exposed subject has one or more symptoms associated with a viral infection, wherein the viral infection is a viral infection resulting from entering or infecting a subject via association with or binding to an ACE2 receptor in the subject, or the viral infection is a coronavirus infection, for example, the viral infection is a COVID-19 infection.

106. The method of any one of claims 82-101of claim 105, wherein the exposed subject has a viral infection, wherein the viral infection is a viral infection resulting from entering or infecting a subject via association with or binding to an ACE2 receptor in the subject, or the viral infection is a coronavirus infection, for example, the viral infection is a COVID-19 infection.

107. The method of any one of claims 82-101of claims 105-106, wherein the method is a therapeutic treatment of the subject having the viral infection.

108. The method of any one of claims 82-107, wherein the effective amount of the composition is administered to the subject 1 time, 2 times, 3 times or 4 times per day, for 1 day, 2 days, 3 days, 4 days, 5 days, 6 days, 1 week, 2 weeks, or 3 weeks.

109. The method of any one of claims 82-108, wherein the amount of the administered composition is a prophylactically effective amount.

110. The method of any one of claims 82-109, wherein the amount of the administered composition is a therapeutically effective amount.

111. The method of any one of claims 82-110, wherein the virus is a virus that enters or infects the subject via association with or binding to an ACE2 receptor in the subject.

112. The method of any one of claims 82-110, wherein the virus is a coronavirus.

113. The method of any one of claims 82-110, wherein the virus is a COVID-19 virus.

114. The method of any one of claims 82-113, wherein the viral infection is a viral infection resulting from entering or infecting a subject via association with or binding to an ACE2 receptor in the subject.

115. The method of any one of claims 82-113, wherein the viral infection is a coronavirus infection.

116. The method of any one of claims 82-113, wherein the viral infection is a COVID-19 infection.
